(19) United States
(12) Patent Application Publication
(10) Pub. No.: US 2015/0079169 A1
(43) Pub. Date: Mar. 19, 2015

(54) CONTROLLED DOSING OF CLOPIDOGREL WITH GASTRIC ACID INHIBITION THERAPIES

(71) Applicant: John R. Plachetka, Chapel Hill, NC (US)

(72) Inventor: John R. Plachetka, Chapel Hill, NC (US)

(73) Assignee: POZEN INC., Chapel Hill, NC (US)

(21) Appl. No.: 14/344,688

(22) PCT Filed: Sep. 14, 2012
(86) PCT No.: PCT/US2012/055574

§ 371 Date: Oct. 23, 2014

Related U.S. Application Data
(60) Provisional application No. 61/534,666, filed on Sep. 14, 2011.

(51) Int. Cl. A61K 31/4365; A61K 31/616; A61K 9/24; A61K 31/4439

(52) U.S. Cl. CPC A61K 31/4365; A61K 31/4439; A61K 31/616; A61K 9/209
USPC 424/471; 514/301; 514/161; 424/468

(57) ABSTRACT

The present invention provides for novel formulations of clopidogrel in combination with proton pump inhibitors (PPI) and optionally with NSAIDs, for use as improved antiplatelet therapies in stroke and cardiovascular indications. The invention provides the delivery of clopidogrel in pulses or waves, such that the total dose is phased/spread out over time and, advantageously, combined with omeprazole in a way to minimize the conflicting actions these two drugs may have on each other. In addition, the invention also provides the delivery of clopidogrel and a PPI, and optionally aspirin, in a sequential (orderly) manner that would allow for the delivery and metabolism of clopidogrel first, followed by the PPI, and thereafter optionally aspirin. A particular mode of the invention involves the combination of clopidogrel with coformulated immediate release omeprazole+enteric coated aspirin.

The application claims priority to U.S. Provisional Patent Application No. 61/534,666 filed Sep. 14, 2011, which is incorporated herein by reference in its entirety.

### BACKGROUND OF THE INVENTION

1. Field of the Invention

The present invention relates to the fields of biology, medicine, and pharmacology. More specifically, the invention provides novel formulations of clopidogrel and a gastric acid inhibitor, optionally with an NSAID, and methods of use therefor.

2. Description of Related Art

Dual antiplatelet therapy (DAPT) with clopidogrel and aspirin presents an effective strategy to reduce ischemic event occurrence in patients treated with coronary artery stents in the presence or absence of an acute coronary syndrome (ACS), but DAPT is associated with increased risk of serious gastrointestinal bleeding (GIB) (King et al., 2008; Moukarbel et al., 2009); with GIB resulting in premature discontinuation of DAPT therapies and a ˜2.5 times increased risk of death in subjects undergoing such treatment regimens (Moukarbel et al., 2009; Bhatt et al., 2008). As a consequence, use of proton pump inhibitors (PPIs) have been recommended and widely adapted in patients with risk (factors) for upper GIB treated with DAPT (Bhatt et al., 2008).

Compared to its use without a PPI, concomitant use of clopidogrel and PPIs has been associated with an attenuated pharmacodynamic effect of clopidogrel and a potential reduction in the clinical benefits of clopidogrel after ACS (Gurbel et al., 2010; Gurbel and Tantry, 2011; Angiolillo et al., 2011; Ferreiro et al., 2010). However, other studies have not supported an effect of PPIs on major cardiovascular outcomes in patients treated with clopidogrel (Gurbel and Tantry, 2011). Despite the lack of consensus on the clinical significance of this drug interaction, both the Food and Drug Administration and the European Medicines Agency have issued warnings about the interaction and have adjusted product information.

Although the precise cause of the pharmacodynamic interaction between clopidogrel and enteric coated PPIs is unknown, reports suggest that insufficient clopidogrel active metabolite generation results from competition of PPIs and clopidogrel for metabolism by cytochrome P450 (CYP) 2C19 (Angiolillo et al., 2011). This has led to the suggestion that separating the dosing of clopidogrel and PPIs would reduce the amount of omeprazole competing for the same enzymatic site as clopidogrel (Laine and Hennekens, 2010). However, several studies have reported that spacing of clopidogrel and enteric coated (EC) omeprazole dosing in healthy volunteers did not lessen the interaction (Angiolillo et al., 2011; Ferreiro et al., 2010). An experimental drug, PA32540 (Pozen Inc., Chapel Hill N.C.) contains omeprazole and enteric-coated aspirin. However, the release mechanisms of PA32540 are associated with a substantially different omeprazole pharmacokinetic profile compared to commercially available (enterically coated) omeprazole, and the effect of PA32540 on clopidogrel's antiplatelet effect is currently unknown (Gurbel et al., 2009). Thus, there remains a need to identify new approaches to the delivery of clopidogrel to subjects in need thereof.

### SUMMARY OF THE INVENTION

The present invention is designed to provide new antiplatelet therapies, particularly those that provide treatments for subjects at risk of secondary cardiovascular events. The treatments are designed to deliver PPIs, such as omeprazole, and clopidogrel in either a coformulation or in simultaneously delivered individual formulations. In addition, the invention provides the delivery of clopidogrel in pulses or waves, such that the total dose is phased/spread out over time and, advantageously, combined with omeprazole in a way to minimize the conflicting actions these two drugs may have on each other. In addition, the invention also provides the delivery of clopidogrel and a PPI, and optionally aspirin, in a sequential (orderly) manner that would allow for the delivery and metabolism of clopidogrel first, followed by the PPI, and thereafter optionally aspirin. A particular mode of the invention involves the combination of clopidogrel with coformulated immediate release omeprazole+enteric coated aspirin. The subject may suffer from or be at risk of stroke, heart attack, arterial stenosis or atherosclerosis, or has undergone or will undergo vein graft transplant or stent placement.

Thus, in accordance with the present invention, there is provided a method of providing an antiplatelet therapy to a subject in need thereof comprising co-administering to said subject a proton pump inhibitor (PPI) and clopidogrel such that (a) said clopidogrel and said omeprazole are delivered in a sequential manner; (b) said clopidogrel is released (i) prior to or (ii) prior to and after said PPI; and (c) said PPI achieves a peak plasma concentration at least 1 hour after a first clopidogrel peak plasma concentration. The clopidogrel may delivered in multiple pulses, such as 2, 3 or 4 pulses.

The PPI may achieve a peak plasma concentration at least 2 hours prior to a second, third or fourth clopidrogrel pulse, at least 4 hours prior to a second, third or fourth clopidrogrel pulse, or at least 6 hours prior to a second, third or fourth clopidrogrel pulse. The subject may further be administered aspirin, such as aspirin formulated for enteric release. The clopidogrel and PPI may be coformulated in a single drug formulation, which may further include aspirin. Such a triple combination may comprise an aspirin core, a PPI layer surrounding said aspirin core, and a clopidogrel layer surrounding said PPI layer. Alternatively, the clopidogrel and the PPI may be formulated individually but administered at the same time. The subject may suffer from or be at risk of stroke, heart attack, arterial stenosis or atherosclerosis, or may have or may undergo vein graft transplant or stent placement.

The PPI may achieve a peak plasma concentration at least 2 hours after the first clopidogrel peak plasma concentration, or may achieve a peak plasma concentration at about 2-6 hours after the first clopidogrel peak plasma concentration. The clopidogrel may be pulsed twice at about 37.5 mg per pulse, or pulsed three times at about 25 mg per pulse. The PPI may be selected from the group consisting of omeprazole, lansoprazole, pantoprazole, esomeprazole, rabeprazole, and dexlansoprazole. In the case of omeprazole, aspirin may be coformulated with said omeprazole and said omeprazole may be delivered prior to said aspirin.

In another embodiment, there is provided a drug formulation comprising (a) clopidogrel, wherein clopidrogrel is released immediately; and (b) a proton pump inhibitor (PPI), wherein said PPI is released subsequent to said clopidogrel. The drug formulation may further comprise aspirin, including enteric release aspirin, and for example, where the drug formulation comprises an aspirin core, an PPI layer surrounding said aspirin core, and a clopidogrel layer surrounding said PPI layer. The PPI may achieve a peak plasma concentration at least 2 hours prior to a second, third or fourth clopidrogrel pulse, or may achieve a peak plasma concentration at least 4 hours prior to a second, third or fourth clopidrogrel pulse, or may achieve a peak plasma concentration at least 6 hours prior to a second, third or fourth clopidrogrel pulse. The PPI may achieve a peak plasma concentration at least 2 hours after the first clopidogrel peak plasma concentration, or may achieve a peak plasma concentration about 2-6 hours after the first clopidogrel peak plasma concentration. The clopidogrel may be delivered in multiple pulses, such as 2, 3 or 4 pulses. The clopidogrel may be pulsed twice at about 37.5 mg per pulse, or pulsed three times at about 25 mg per pulse. The total 24 hour dose of clopidogrel is 75-300 mg. The PPI may be selected from the group consisting of omeprazole, lansoprazole, pantoprazole, esomeprazole, rabeprazole, and dexlansoprazole. The dose of omeprazole may be 20-40 mg. Aspirin may be coformulated with said omeprazole, wherein said omeprazole is delivered prior to said aspirin.

In yet another embodiment, there is provided a method of administering a proton pump inhibitor PPI and a second agent to a subject such that (a) said PPI is delivered in at least two pulses; and (b) said second agent is delivered prior to at least a second PPI pulse, wherein said second agent interacts with CYP2C19. The second agent may be an antidepressant, a barbiturate, a proton pump inhibitor, an antimalarial drug or a antitumor drug. The first pulse of the PPI may be delivered immediately. The subject may suffer from or is at risk of stroke, heart attack, arterial stenosis or atherosclerosis, or has or will undergo vein graft transplant or stent placement. The second agent may be selected from the group consisting of clopidogrel, phenyloin, tamoxifen, tolbutamide, torsemide, fluvastatin, warfarin, heparin, ardeparin, dalteparin, danaparoid, enoxaparin, tinzaparin, anistreplase, dipyridamole, streptokinase, ticlopidine and urokinase. The second agent may be an antidepressant, a barbiturate, a proton pump inhibitor, an antimalarial drug or a antitumor drug. The PPI is selected from the group consisting of omeprazole, lansoprazole, pantoprazole, esomeprazole, rabeprazole, and dexlansoprazole. In particular, the PPI may be omeprazole and the second agent may be clopidogrel, optionally wherein (a) omeprazole is delivered in two 20 mg pulses, and clopidogrel is delivered as a single 75 mg pulse, and/or (b) said omeprazole is delivered in two pulses, one provided immediately and one provided substantially after 2 hours, and said clopidogrel is delivered within about 2 hours of administration. The second agent may be substantially delivered within 2 hours, and the second pulse of said PPI may be substantially delivered after 2 hours. The method may further comprise providing aspirin to said subject.

Yet a further embodiment comprises a drug formulation comprising (a) a PPI delivered in at least two pulses; and (b) a second agent delivered prior to at least a second PPI pulse, wherein said second agent interacts with CYP2C19. The drug formulation may deliver a first pulse of said PPI immediately. The drug formulation may delivers the PPI in two pulses, one provided immediately and one substantially after 2 hours, and the drug formulation may deliver clopidogrel within about 2 hours of administration. The second agent may be selected from the group consisting of clopidogrel, phenyloin, tamoxifen, tolbutamide, torsemide, fluvastatin, warfarin, heparin, ardeparin, dalteparin, danaparoid, enoxaparin, tinzaparin, anistreplase, dipyridamole, streptokinase, ticlopidine and urokinase. The second agent may be an antidepressant, a barbiturate, a proton pump inhibitor, an antimalarial drug or a antitumor drug. The PPI may be selected from the group consisting of omeprazole, lansoprazole, pantoprazole, esomeprazole, rabeprazole, and dexlansoprazole. In a particular case, the PPI is omeprazole and said second agent is clopidogrel. The omeprazole is formulated for delivery in two 20 mg pulses, and clopidogrel is formulated for delivery as a single 75 mg pulse, and/or the omeprazole is delivered in two pulses, one provided immediately and one provided substantially after 2 hours, and said clopidogrel is delivered within about 2 hours of administration. The formulation may substantially deliver the second agent within 2 hours, and said formulation may substantially deliver a second pulse of said PPI after 2 hours. The drug formulation may further comprise aspirin, such as aspirin formulated for enteric delivery.

In still a further embodiment, there is provided a method of treating a subject with an antiplatelet therapy comprising administering to said subject (a) enteric-coated aspirin coformulated with immediate-release omeprazole; and (b) clopidogrel, wherein (a) and (b) are dosed at least 10 hours apart. The aspirin may be dosed at 325 mg and omeprazole is dosed at 20-40 mg. The clopidogrel may be dosed after aspirin/omeprazole, and/or dosed at 75-300 mg. The subject may be treated daily with both (a) and (b).

Also provided are uses of PPIs, such as omeprazole, and clopidogrel in either a coformulation or in simultaneously delivered individual formulations for the provision of anti-platelet therapies, such as those involving secondary cardiovascular events, and further as described in each of the methods above.

The embodiments in the Examples section are understood to be embodiments of the invention that are applicable to all aspects of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Components of PA32540 Tablet.

FIG. 2. SPACING study design. ECASA=enteric coated aspirin, C=Clopidogrel

FIG. 3. ΔPA₂₀ₘₐₓ by Time and Treatment.

FIG. 4. ΔPA₅ₘₐₓ by Time and Treatment.

FIG. 5. ΔPRU by Time and Treatment.

FIG. 6. ΔPRI by Time and Treatment.

FIG. 7. PK Profile of Standard Clopidogrel versus Two and Three Pulsed Clopidogrel.

### DESCRIPTION OF ILLUSTRATIVE EMBODIMENTS

Clopidogrel is a commonly used anti-platelet drug for the prevention of vascular ischemic events, other acute coronary diseases, and coronary procedures. Clopidogrel acts by irreversibly binding/blocking specific ADP receptors on the circulating platelets which in turn inhibit their aggregation and cross linking. Platelets are regenerated continuously and, therefore, a single immediate release dose of clopidogrel will lose its pharmacological effect once the plasma level of the active drug dissipates. Clopidogrel is a pro-drug and is metabolized by liver enzymes into its pharmacologically active component. The pharmacological effect of clopidogrel has been reported to be decreased if it is taken with other drugs that share the same metabolic pathway in the liver.

Thus, the field has recognized a problem with regard to an unfavorable interaction between clopidogrel and PPIs. The present invention seeks to solve this problem in at least one of three ways, or a combination thereof. First, by delaying the release of clopidogrel as compared to the PPI, which optionally can be formulated for immediate delivery, one can separate the delivery of each drug and reduce the apparent competition for CYP2C19. Second, one can deliver clopidogrel in pulses or waves, thereby achieving multiple plasma peak deliveries while decreasing plasma peak concentrations of clopidogrel at any point. Again, this can be coupled with immediate release PPI. Optionally, the co-delivery of aspirin may be included. Third, one can deliver clopidogrel first when co-delivered with PPI to allow for exposure of clopidogrel to the liver enzymes prior to exposure to competing PPI.

As discussed in the Examples that follow, an experimental drug containing aspirin and omeprazole, designated PA32540 (Pozen Inc., Chapel Hill N.C.), is the subject of the SPACING (Spaced PA32540 with Clopidogrel INteraction Gauging (SPACING)) Study. This study was designed to evaluate whether platelet inhibition during dual antiplatelet therapy with PA32540 and clopidogrel (Plavix®, Sanofi-Aventis U.S., Bridgewater N.J.), administered synchronously or spaced 10 hours apart, was non-inferior to a strategy of synchronous administration of 325 mg EC aspirin and clopidogrel. As explained below, the drug was in fact found non-inferior.

Thus, in order to overcome the aforementioned limitations on co-delivery of clopidogrel and PPI's, the present invention provides solid dosage forms that can deliver two or more smaller doses of clopidogrel at the same total dose as commercially available products, but separated sufficiently to avoid the unfavorable drug interactions of clopidogrel with PPIs. In addition, the present invention provides solid dosage forms that can sequentially deliver clopidogrel, omeprazole, and aspirin. These and other aspects of the invention are described in detail below.

### I. Clopidogrel

Clopidogrel is an oral, thienopyridine class antiplatelet agent used to inhibit blood clots in coronary artery disease, peripheral vascular disease, and cerebrovascular disease. It is marketed by Bristol-Myers Squibb and Sanofi-Aventis under the trade name Plavix®. Adverse effects include hemorrhage, severe neutropenia, and thrombotic thrombocytopenic purpura (TTP).

Clopidogrel is a prodrug, the action of which may be related to an adenosine diphosphate (ADP) receptor on platelet cell membranes. The drug specifically and irreversibly inhibits the P2Y12 subtype of ADP receptor, which is important in aggregation of platelets and cross-linking by the protein fibrin. The blockade of this receptor inhibits platelet aggregation by blocking activation of the glycoprotein IIb/IIIa pathway. The IIb/IIIa complex functions as a receptor mainly for fibrinogen and vitronectin but also for fibronectin and von Willebrand factor. Activation of this receptor complex is the “final common pathway” for platelet aggregation and is important in the cross-linking of platelets by fibrin. At least some platelet inhibition can be demonstrated two hours after a single dose of oral clopidogrel, but the onset of action is slow, so that a loading-dose of 300-600 mg is usually administered.

Due to opening of the thiophene ring, the metabolite chemical structure has three sites of chirality, making a total of eight possible isomers. These are: (a) a stereocentre at C4 (attached to the —SH thiol group), (b) a stereobond at C3-C16 double-bound and (c) the original stereocenter at C7. Only one of the eight structures is an active antiplatelet drug. This has the following configuration; a (Z) configuration at C3-C16 double-bound, the original (S) configuration stereocenter at C7 and although the stereocentre at C4 cannot be directly determined (the thiol group is too reactive), work with the active metabolite of the related drug Prasugrel suggests that the (R)-configuration of the C4 group is critical for P2Y12 and platelet-inhibitory activities.

Clopidogrel is indicated for:
prevention of vascular ischemic events in patients with symptomatic atherosclerosis
acute coronary syndrome without ST-segment elevation (NSTEMI)
ST elevation MI (STEMI)
   It is also used, along with aspirin, for the prevention of thrombosis after placement of intracoronary stent or as an alternative antiplatelet drug for patients who are intolerant to aspirin.

Clopidogrel is marketed as clopidogrel bisulfate (clopidogrel hydrogen sulfate), most commonly under the trade name Plavix, as 75 mg oral tablets. After repeated 75 mg oral doses of clopidogrel (base), plasma concentrations of the parent compound, which has no platelet inhibiting effect, are very low and are generally below the quantification limit (0.000258 mg/L) beyond two hours after dosing. Following an oral dose of ¹⁴C-labeled clopidogrel in humans, approximately 50% was excreted in the urine and approximately 46% in the feces in the five days after dosing.

Administration of clopidogrel bisulfate with meals did not significantly modify the bioavailability of clopidogrel as assessed by the pharmacokinetics of the main circulating metabolite. Clopidogrel is rapidly absorbed after oral administration of repeated doses of 75 mg clopidogrel (base), with peak plasma levels (approx. 3 mg/L) of the main circulating metabolite occurring approximately one hour after dosing. The pharmacokinetics of the main circulating metabolite are linear (plasma concentrations increased in proportion to dose) in the dose range of 50 to 150 mg of clopidogrel. Absorption is at least 50% based on urinary excretion of clopidogrel-related metabolites. Clopidogrel and the main circulating metabolite bind reversibly in vitro to human plasma proteins (98% and 94%, respectively). The binding is nonsaturable in vitro up to a concentration of 110 mg/mL. In vitro and in vivo, clopidogrel undergoes rapid hydrolysis into its carboxylic acid derivative. In plasma and urine, the glucuronide of the carboxylic acid derivative is also observed.

Clopidogrel is a pro-drug activated in the liver by cytochrome P450 enzymes, including CYP2C19. The active metabolite has an elimination half-life of about eight hours and acts by forming a disulfide bridge with the platelet ADP receptor. Several recent landmark studies have proven the importance of CYP2C19 genotyping in treatment using clopidogrel or Plavix. In March 2010, the U.S. FDA placed a Box Warning on Plavix to make patients and healthcare providers aware that CYP2C19 poor metabolizers, representing up to 14% of patients, are at high risk of treatment failure and that testing is available. Researchers have found that patients with variants in cytochrome P-450 2C19 (CYP2C19) have lower levels of the active metabolite of clopidogrel, less inhibition of platelets, and a 3.58-fold greater risk for major adverse cardiovascular events such as death, heart attack, and stroke; the risk was greatest in CYP2C19 poor metabolizers. CYP2C19 is an important drug-metabolizing enzyme that catalyzes the biotransformation of many clinically useful drugs including antidepressants, barbiturates, proton pump inhibitors, antimalarial and antitumor drugs. Clopidogrel is one of the drugs metabolized by this enzyme.

Serious adverse drug reactions associated with clopidogrel therapy include:
severe neutropenia (low white blood cells) (incidence: 1/2,000)
thrombotic thrombocytopenic purpura (TTP) (incidence: 4/1,000,000 patients treated)
hemorrhage (the annual incidence of hemorrhage may be increased by the co-administration of aspirin)
gastrointestinal hemorrhage (incidence: 2.0% annually)
cerebral hemorrhage (incidence: 0.1 to 0.4% annually)
   Use of non-steroidal anti-inflammatory drugs is discouraged in those taking clopidogrel due to increased risk of digestive tract hemorrhage (Diener et al., Lancet 364-331-7, 2004).

Clopidogrel interacts with the following drugs: proton pump inhibitors, phenyloin (Dilantin); tamoxifen (Nolvadex); tolbutamide (Orinase); torsemide (Demadex); fluvastatin (Lescol); a blood thinner such as warfarin (Coumadin), heparin, ardeparin (Normiflo), dalteparin (Fragmin), danaparoid (Orgaran), enoxaparin (Lovenox), or tinzaparin (Innohep); Tissue Plasminogen Activator (Activase), anistreplase (Eminase), dipyridamole (Persantine), streptokinase (Kabikinase, Streptase), ticlopidine (Ticlid), and urokinase (Abbokinase). In November 2009, the FDA announced that clopidogrel should not be taken with CYP2C19 inhibitors as omeprazole and esomeprazole.

Clopidogrel is effective at reducing cardiovascular events in people at high risk due to previous CVD. Clopidogrel is effective in reducing a combined outcome of major cardiovascular events (MI, ischaemic stroke, vascular death) in people with MI, stroke, or peripheral artery disease. Thienopyridines like clopidogrel, compared with aspirin, may decrease gastrointestinal haemorrhage but increase the risk of skin rash or diarrhea. One study of 19,185 people with a history of MI, stroke, or peripheral arterial disease compared clopidogrel (75 mg daily) versus aspirin (325 mg daily) and found that clopidogrel significantly reduced the risk of major cardiovascular events (defined as ischaemic stroke, MI, or vascular death: average rate per year 5% (939 events/17,636 patient-years at risk) with clopidogrel v. 6% (1021 events/17,519 patient-years at risk) with aspirin; RRR 8.7%, 95% CI 0.30% to 16.5%; P=0.04). Another study showed that ticlopidine or clopidogrel modestly but significantly reduced cardiovascular events compared with aspirin (OR 0.91, 95% CI 0.84 to 0.98; average 11 events prevented/1000 people treated with a thienopyridine instead of aspirin for 2 years, 95% CI; 2 events prevented/1000 people treated to 19 events prevented/1000 people treated).

### II. Proton Pump Inhibitor/NSAID Formulations

A. PPI's

Proton pump inhibitors (PPIs) are drugs whose main action is a pronounced and long-lasting reduction of gastric acid production. They are the most potent inhibitors of acid secretion available today. The group followed and has largely superseded another group of pharmaceuticals with similar effects, but different mode-of-action, called H2-receptor antagonists. PPIs are among the most widely-selling drugs in the world and are generally considered effective. The vast majority of these drugs are benzimidazole derivatives; however, promising new research indicates that imidazopyridine derivatives may be a more effective means of treatment. High dose or long-term use of PPIs carry a possible increased risk of bone fractures.

PPIs are utilized in the treatment of many conditions such as:
dyspepsia
peptic ulcer disease (PUD)
gastroesophageal reflux disease (GORD/GERD)
laryngopharyngeal reflux
Barrett's esophagus
prevention of stress gastritis
gastrinomas and other conditions that cause hypersecretion of acid
Zollinger-Ellison syndrome

Proton pump inhibitors act by irreversibly blocking the hydrogen/potassium adenosine triphosphatase enzyme system (the H+/K+ ATPase, or more common gastric proton pump) of the gastric parietal cells. The proton pump is the terminal stage in gastric acid secretion, being directly responsible for secreting H+ ions into the gastric lumen, making it an ideal target for inhibiting acid secretion. “Irreversibility” refers to the effect on a single copy of the enzyme; the effect on the overall human digestive system is reversible, as the enzymes are naturally destroyed and replaced. Targeting the terminal step in acid production, as well as the irreversible nature of the inhibition, results in a class of drugs that is significantly more effective than H2 antagonists and reduces gastric acid secretion by up to 99%.

The higher pH in the stomach due to PPI therapy will aid in the healing of duodenal ulcers, and reduces the pain from indigestion and heartburn, which can be exacerbated by stomach acid. However, lack of stomach acid is also called hypochlorhydria, the lack of sufficient hydrochloric acid, or HCl. Hydrochloric acid is required for the digestion of proteins and for the absorption of nutrients, particularly of vitamin B12 and of calcium.

Proton pump inhibitors are given in an inactive form. The inactive form is neutrally charged (lipophilic) and readily crosses cell membranes into intracellular compartments (like the parietal cell canaliculus) that have acidic environments. In an acid environment, the inactive drug is protonated and rearranges into its active form. As described above, the active form will covalently and irreversibly bind to the gastric proton pump, inactivating it.

In general, the absorption of proton pump inhibitors is unaffected by co-administration with food. The rate of omeprazole absorption, however, is decreased by concomitant food intake. In addition, the absorption of lansoprazole and esomeprazole is decreased and delayed by food, but it has been suggested that these pharmacokinetic effects have no significant impact on efficacy. The elimination half-life of proton pump inhibitors ranges from 0.5-2 hours, however the effect of a single dose on acid secretion usually persists up to 2-3 days. This is because of accumulation of the drug in parietal cell canaliculi and the irreversible nature of proton pump inhibition.

Clinically used proton pump inhibitors:
Omeprazole (Losec®, Prilosec®, Zegerid®, Lomac®, Omepral®, Omez®)
Lansoprazole (Prevacid®, Zoton®, Inhibitol®, Levant®, Lupizole®)
Dexlansoprazole (Kapidex®, Dexilant®)
Esomeprazole (Nexium®, Esotrex®)
Pantoprazole (Protonix®, Somac®, Pantoloc®, Pantozol®, Zurcal®, Zentro®, Pang)
Rabeprazole (Zechin®, Rabecid®, Nzole-D®, AcipHex®, Pariet®, Rabeloc®)
   In general, proton pump inhibitors are well tolerated, and the incidence of short-term adverse effects is relatively uncommon. The range and occurrence of adverse effects are similar for all of the proton pump inhibitors, though they have been reported more frequently with omeprazole. This may be due to its longer availability and, hence, clinical experience. Common adverse effects include: headache, nausea, diarrhea, abdominal pain, fatigue, and dizziness.

A recent study has also suggested that proton pump inhibitors significantly decreased the effect of clopidogrel on platelets as tested by VASP phosphorylation. The clinical impact of these results must be assessed by further investigations, but a PPI treatment should not be added to the antiplatelet dual therapy without formal indication.

B. Aspirin

Nonsteroidal anti-inflammatory drugs (NSAIDs) are drugs with analgesic and antipyretic (fever-reducing) effects and which have, in higher doses, anti-inflammatory effects. The term “nonsteroidal” is used to distinguish these drugs from steroids, which, among a broad range of other effects, have a similar eicosanoid-depressing, anti-inflammatory action. As analgesics, NSAIDs are unusual in that they are non-narcotic.

Most NSAIDs act as nonselective inhibitors of the enzyme cyclooxygenase (COX), inhibiting both the cyclooxygenase-1 (COX-1) and cyclooxygenase-2 (COX-2) isoenzymes. COX catalyzes the formation of prostaglandins and thromboxane from arachidonic acid (itself derived from the cellular phospholipid bilayer by phospholipase A2). Prostaglandins act (among other things) as messenger molecules in the process of inflammation. Many aspects of the mechanism of action of NSAIDs remain unexplained, and for this reason further COX pathways are hypothesized. The COX-3 pathway was believed to fill some of this gap but recent findings make it appear unlikely that it plays any significant role in humans and alternative explanation models are proposed.

The widespread use of NSAIDs has meant that the adverse effects of these drugs have become increasingly prevalent. The two main adverse drug reactions (ADRs) associated with NSAIDs relate to gastrointestinal (GI) effects and renal effects of the agents. These effects are dose-dependent, and in many cases severe enough to pose the risk of ulcer perforation, upper gastrointestinal bleeding, and death, limiting the use of NSAID therapy. An estimated 10-20% of NSAID patients experience dyspepsia, and NSAID-associated upper gastrointestinal adverse events are estimated to result in 103,000 hospitalizations and 16,500 deaths per year in the United States, and represent 43% of drug-related emergency visits. NSAIDs, like all drugs, may interact with other medications. For example, concurrent use of NSAIDs and quinolones may increase the risk of quinolones' adverse central nervous system effects, including seizure.

In people with known vascular disease, aspirin is additionally known to reduce the incidence of non-fatal myocardial infarction, non-fatal stroke and vascular death by about a quarter. Aspirin has been shown to result in a reduction of coronary events, and also reduces the risk of ischemic stroke. Aspirin not only reduces the re-occurrence of vascular catastrophes, but probably also resulted in lower death rates. Unfortunately, aspirin also increases the risk for GI ulcers. This effect is present in both primary and secondary prevention trials. Most cardiovascular risk patients receive not only aspirin for secondary prevention of vascular disease, but also other interventions such as blood pressure control medications and statins.

It is expected that a skilled pharmacologist may adjust the amount of aspirin in a pharmaceutical composition or administered to a patient based upon standard techniques well known in the art. However, aspirin will typically be present in tablets or capsules in an amount of between about 50 mg and 1000 mg, including 75 mg, 81.25 mg, 100 mg, 150 mg, 162.5 mg, 250 mg, 300 mg, 325 mg, 400 mg, 500 mg, 650 mg, 800 mg and 1000 mg. Typical daily dosages will be in an amount ranging from 500 mg to about 10 g for analgesia or inflammation, and in an amount ranging from 50 mg to 500 mg for secondary prevention of cardiovascular disease.

C. NSAID/PPI Combinations

U.S. Pat. No. 6,926,907, incorporated herein by reference, describes the advantageous coformulation of NSAIDs and PPIs. In particular, the invention discussed therein is directed to drug dosage forms that release an agent that raises the pH of a patient's gastrointestinal tract, e.g., a PPI, followed by a non-steroidal anti-inflammatory drug. The dosage form is designed so that the NSAID is not released until the intragastric pH has been raised to a safe level. The invention also encompasses methods of treating patients by administering this coordinated release, gastroprotective combination unit dosage form to achieve pain and symptom relief with a reduced risk of developing gastrointestinal damage such as ulcers, erosions and hemorrhages.

A specific form of this combination is called Vimovo®, which is marketed by Astrazenca. Vimovo® is a combination product that contains naproxen and esomeprazole. It is indicated for signs and symptoms of osteoarthritis, rheumatoid arthritis and ankylosing spondylitis while decreasing the risk of developing NSAID-associated gastric complications. A particular aspect of Vimovo® action is delayed release of naproxen. It is provided in two oral administration forms:
375 mg enteric coated naproxen and 20 mg esomeprazole (as magnesium hydrate); or
500 mg enteric coated naproxen and 20 mg esomeprazole (as magnesium hydrate)

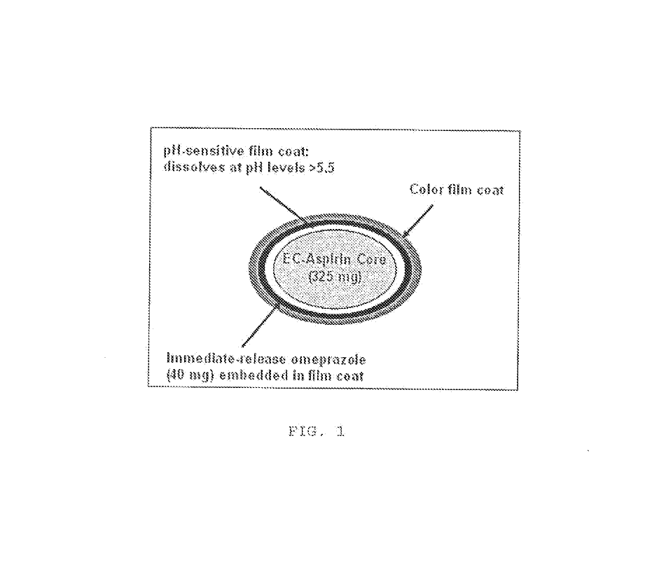

Another specific form of this combination is formulation, PA32540 (Pozen Inc., Chapel Hill N.C.), a tablet containing 325 mg enteric coated (EC) aspirin and 40 mg omeprazole. It is designed to reduce aspirin-related gastrointestinal toxicity while delivering a bioequivalent dose of aspirin. This tablet is unique in that omeprazole is not EC (delayed release formulation) or buffered as it is in other PPI products (Grubel et al., 2009). Instead, omeprazole is contained in the outer layer of the PA32540 tablet in an immediate release form, available for rapid dissolution (FIG. 1). Its therapeutic activity is rapid and occurs prior to the dissolution of the aspirin component contained within the core of the multi-layered tablet (Grubel et al., 2009). To further ensure the sequential delivery of the two components, the aspirin core is coated by polymers which prevent dissolution until the pH of the surrounding environment is >5.5. A bioequivalence study (PA32540-104) demonstrated that, with respect to salicylic acid pharmacokinetics, PA32540 is bioequivalent to commercially available 325 mg EC aspirin (Fort et al., 2008). Compared to 81 mg EC aspirin, PA32540 was associated with greater inhibition of in vivo thromboxane generation and no greater upper gastrointestinal damage by Lanza score (Grubel et al., 2009).

### III. Formulations

A. Dual Delivery Systems

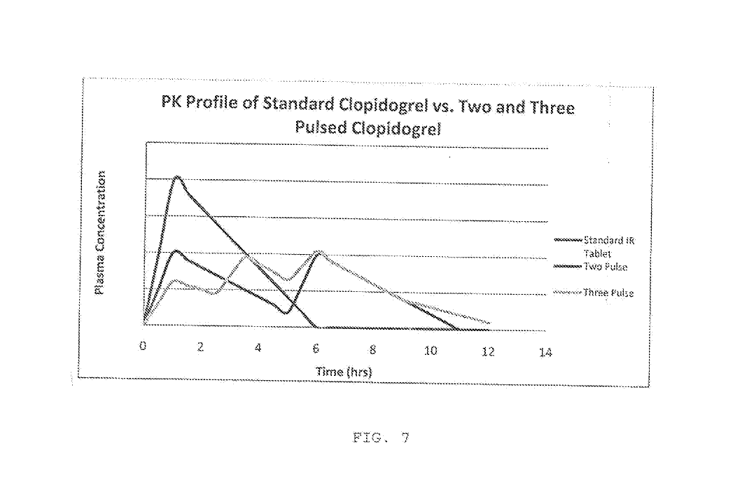

In a first approach, one may use distinct dosage forms to simultaneously deliver clopidogrel and a PPI to a patient. In general, the goal is to spread the clopidogrel delivery over about 1 to 12 hours, and to have multiple clopidogrel plasma pulses (defined as multiple peaks in plasma level concentration separated from each other) separated from the earlier release of PPI. This increases the duration of platelet inhibition by extending the duration of the plasma exposure of clopidogrel, while concomitantly decreasing clopidogrel's potential to interact with the CYP2C19-metabolized PPI's by reducing the initial dose of clopidogrel. The follow-on doses will be exposed to the liver enzymes about 1-12 hours after the initial dose, therefore, avoiding competition with PPI's. These formulations can be used advantageously with drug formulations as described in U.S. Pat. No. 6,926,907, and in particular, those discussed above such as Vimovo® and PA32540. In such situations, the drugs and dosings will be provided to achieve a separation of PPI and one or more of the clopidogrel peak releases by 3 or more hours, 6 or more hours, 9 or more hours, 10 or more hours, 11 or more hours or about 12 hours, including ranges such as 3-6 hours, 6-9 hours, 9-12, hours, 6-12 hours, 3-9 hours and 3-12 hours. A comparison of a multi-pulse delivery of clopidogrel to standard clopidogrel is shown in FIG. 7.

The following is a discussion of various clopidogrel formulations which can achieve the aforementioned goals, without limiting the possible combinations.

1. Tablet in Tablet/Multilayered Tablet

In one version, the formulation employs a “tablet in a tablet” or “multilayer tablet” form. This comprises clopidogrel inner core coated with an enteric polymer that is pH sensitive. In general, the desired release range will be about pH 5-7.5. For example, Eudragit (Methyl Acrylic Acid) L30D-55 permits release of drug when pH is greater than 5, Aquoat (Hypermellose Acetate Succinate) M grade permits release of drug when pH is greater than 6, and Eudragit (Methyl Acrylic Acid) FS 30D or S-100 permit release of drug when pH is greater than 7. An immediate release portion containing clopidogrel is compressed around the coated core. The coated clopidogrel core is then spray-coated with an immediate release portion containing omeprazole.

2. Multi-Tablet Capsule

A multi-tablet capsule approach would start with multiple tablets having an immediate release core of clopidogrel, each of which is coated with a distinct enteric polymer that is pH sensitive. For example, Eudragit (Methyl Acrylic Acid) L30D-55 permits release of drug when pH is greater than 5, Aquoat (Hypermellose Acetate Succinate) M grade permits release of drug when pH is greater than 6, and Eudragit (Methyl Acrylic Acid) FS 30D or S-100 permit release of drug when pH is greater than 7. Two or more different clopidogrel tablets having different release profiles are then encapsulated along with an immediate release omeprazole tablet.

3. Multi-Particulate Capsules

Multiple clopidogrel and omeprazole beads are enclosed in a capsule where beads are coated with a distinct enteric polymer. For example, Eudragit (Methyl Acrylic Acid) L30D-55 permits release of drug when pH is greater than 5, Aquoat (Hypermellose Acetate Succinate) M grade permits release of drug when pH is greater than 6, and Eudragit (Methyl Acrylic Acid) FS 30D or S-100 permits release of drug when pH is greater than 7. Pulsed delivery of clopidogrel with immediate omeprazole can be achieved by encapsulating two or more types of clopidogrel beads (immediate release, enteric release) along with immediate release ompeprazole beads.

4. Multi-Particulate Tablets

Multi-particulate tablets include multiple clopidogrel and omeprazole beads compressed into a tablet where each bead is coated with a distinct enteric polymer. For example, Eudragit (Methyl Acrylic Acid) L30D-55 permits release of drug when pH is greater than 5, Aquoat (Hypermellose Acetate Succinate) M grade permits release of drug when pH is greater than 6, and Eudragit (Methyl Acrylic Acid) FS 30D or S-100 permit release of drug when pH is greater than 7. Pulsed delivery of clopidogrel with immediate release omeprazole can be achieved by compressing two or more types of clopidogrel beads within a matrix of immediate release clopidogrel and omeprazole powder and/or granule blend into a single tablet.

B. Three Drug Combinations

In another embodiment, the invention encompasses novel drug formulations that permit the concurrent dosing of clopidogrel with NSAID/PPI delivery. As above, the goal is to spread the clopidogrel delivery over about 1 to 12 hours, and to have multiple clopidogrel plasma concentration peaks separated from each other and from the earlier release of PPI. This increases the duration of platelet inhibition by extending the duration of the plasma exposure of clopidogrel, while concomitantly decreasing clopidogrel's potential to interact with the CYP2C19-metabolized PPIs by reducing the initial dose of clopidogrel. The follow-on doses will be exposed to the liver enzymes about 1-12 hours after the initial dose, therefore, avoiding competition with PPI's. These formulations will provide separation of PPI and one or more clopidogrel peak releases by 3 or more hours, 6 or more hours, 9 or more hours, 10 or more hours, 11 or more hours or about 12 hours, including ranges such as 3-6 hours, 6-9 hours, 9-12, hours, 6-12 hours, 3-9 hours and 3-12 hours.

The following is a discussion of various formulations which can achieve the aforementioned goals, without limiting the possible formulations.

1. Concentric Compressed Tablet

A core of clopidogrel is compression coated with aspirin, possibly separated by a filmcoat. This combined core is then then coated with an enteric polymer that is pH sensitive. For example, Eudragit (Methyl Acrylic Acid) L30D-55 permits release of drug when pH is greater than 5, Aquoat (Hypermellose Acetate Succinate) M grade permits release of drug when pH is greater than 6, and Eudragit (Methyl Acrylic Acid) FS 30D or S-100 permit release of drug when pH is greater than 7. A PPI is then sprayed onto the tablet in filmcoat, or compression coated onto the tablet.

2. Coated Bilayer Tablet

A bilayer tablet comprised of clopidogrel and aspirin is coated with an enteric polymer that is pH sensitive. For example, Eudragit (Methyl Acrylic Acid) L30D-55 permits release of drug when pH is greater than 5, Aquoat (Hypermellose Acetate Succinate) M grade permits release of drug when pH is greater than 6, and Eudragit (Methyl Acrylic Acid) FS 30D or S-100 permit release of drug when pH is greater than 7. PPI is then sprayed or compression coated on the outside of the tablet.

3. Multi-Particulate Capsule

Multiple particles or beads of clopidogrel and aspirin are coated with an enteric polymer that is pH sensitive. For example, Eudragit (Methyl Acrylic Acid) L30D-55 permits release of drug when pH is greater than 5, Aquoat (Hypermellose Acetate Succinate) M grade permits release of drug when pH is greater than 6, and Eudragit (Methyl Acrylic Acid) FS 30D or S-100 permit release of drug when pH is greater than 7. These are then distributed into a capsule containing immediate release beads, slugs, or granules of omeprazole.

4. Multi-Tablet Capsule

A multi-tablet capsule approach consists of immediate release cores of clopidogrel, aspirin, and omeprazole tablets. Clopidogrel and aspirin cores are coated with a distinct enteric polymer that is pH sensitive. For example, Eudragit (Methyl Acrylic Acid) L30D-55 permits release of drug when pH is greater than 5, Aquoat (Hypermellose Acetate Succinate) M grade permits release of drug when pH is greater than 6, and Eudragit (Methyl Acrylic Acid) FS 30D or S-100 permit release of drug when pH is greater than 7. The enteric coated clopidogrel, enteric coated aspirin, and the immediate release omeprazole cores are then enclosed in one capsule.

5. Multi-Particulate Tablet

A multi-particulate tablet approach would start with immediate release beads of clopidogrel and aspirin. Clopidogrel and aspirin beads are coated with a distinct enteric polymer that is pH sensitive. For example, Eudragit (Methyl Acrylic Acid) L30D-55 permits release of drug when pH is greater than 5, Aquoat (Hypermellose Acetate Succinate) M grade permits release of drug when pH is greater than 6, and Eudragit (Methyl Acrylic Acid) FS 30D or S-100 permit release of drug when pH is greater than 7. One would then compress the enteric coated clopidogrel and enteric coated aspirin beads within a matrix of immediate release powder and/or granule blend into a single tablet.

C. Sequential Release Three Combination Tablets

In another embodiment, the invention encompasses novel drug formulations that permit the concurrent dosing of clopidogrel with NSAID and PPI delivery. The goal is to deliver the entire dose of clopidogrel immediately after ingestion of a three combination tablet. In addition to immediately releasing the clopidogrel, the three combination tablet will sequentially deliver an immediate release dose of PPI after the majority of clopidogrel dose has been delivered. Asprin will be delivered after both clopidogrel and PPI have been sequentially delivered in this three combination tablet. This sequential delivery allows for clopidogrel to be exposed the CYP2C19 enzyme first, therefore, clopidogrel metabolism is not impeded or is subject to minimum competition from omeprazole. This three combination tablet will provide separation of clopidogrel and PPI from liver enzyme competition.

The following is a discussion of various formulations which can achieve the aforementioned goals, without limiting the possible formulations.

1. Multi-Layered Tablet

A core of aspirin is enteric coated. Then an immediate release layer of omeprazole is spray coated on the enteric coated aspirin tablet. Then an immediate layer of clopidogrel is spray coated over the omeprazole layer, possibly separated by non-release controlling film coats. This will allow for sequential delivery of clopidogrel, omeprazole, and aspirin.

2. Coated Bilayer Tablet

A bilayer tablet comprised of omeprazole and aspirin is coated with an enteric polymer that is pH sensitive. For example, Eudragit (Methyl Acrylic Acid) L30D-55 permits release of drug when pH is greater than 5, Aquoat (Hypermellose Acetate Succinate) M grade permits release of drug when pH is greater than 6, and Eudragit (Methyl Acrylic Acid) FS 30D or S-100 permit release of drug when pH is greater than 7. Clopidogrel is then sprayed or compression coated on the outside of the tablet.

3. Concentric Compressed Tablet

A core of aspirin is compression coated with omeprazole, possibly separated by a filmcoat. This combined core is then coated with outer layer of immediate release clopidogrel. This allows for sequential delivery of clopidogrel and omeprazole/aspirin.

### IV. Diseases States

The formulations of the present invention are designed in general for antiplatelet (AP) therapies. AP therapies find use in a variety or cardiovascular risk situations, such as stroke, heart attack, arterial stenosis, vein graft transplant, atherosclerosis and stent placement. The following is a brief discussion of these states.

A. Stroke

A stroke, also known as a cerebrovascular accident (CVA), is the rapidly developing loss of brain function(s) due to disturbance in the blood supply to the brain. This can be due to ischemia (lack of blood flow) caused by blockage (thrombosis, arterial embolism), or a hemorrhage (leakage of blood). As a result, the affected area of the brain is unable to function, leading to inability to move one or more limbs on one side of the body, inability to understand or formulate speech, or an inability to see one side of the visual field.

A stroke is a medical emergency and can cause permanent neurological damage, complications, and lead to death. It is the leading cause of adult disability in the United States and Europe and it is the second leading cause of death worldwide. Risk factors for stroke include advanced age, hypertension (high blood pressure), previous stroke or transient ischemic attack (TIA), diabetes, high cholesterol, cigarette smoking and atrial fibrillation. High blood pressure is the most important modifiable risk factor of stroke.

An ischemic stroke is occasionally treated in a hospital with thrombolysis (also known as a “clot buster”), and some hemorrhagic strokes benefit from neurosurgery. Treatment to recover any lost function is stroke rehabilitation, ideally in a stroke unit and involving health professions such as speech and language therapy, physical therapy and occupational therapy. Prevention of recurrence may involve the administration of antiplatelet drugs such as aspirin and dipyridamole, control and reduction of hypertension, and the use of statins. Selected patients may benefit from carotid endarterectomy and the use of anticoagulants.

Strokes can be classified into two major categories: ischemic and hemorrhagic. Ischemic strokes are those that are caused by interruption of the blood supply, while hemorrhagic strokes are those which result from rupture of a blood vessel or an abnormal vascular structure. About 87% of strokes are caused by ischemia, and the remainder by hemorrhage. Some hemorrhages develop inside areas of ischemia (“hemorrhagic transformation”). It is unknown how many hemorrhages actually start as ischemic stroke.

B. Myocardial Infarction

Myocardial infarction (MI) or acute myocardial infarction (AMI), commonly known as a heart attack, is the interruption of blood supply to a part of the heart, causing heart cells to die. This is most commonly due to occlusion (blockage) of a coronary artery following the rupture of a vulnerable atherosclerotic plaque, which is an unstable collection of lipids (fatty acids) and white blood cells (especially macrophages) in the wall of an artery. The resulting ischemia (restriction in blood supply) and oxygen shortage, if left untreated for a sufficient period of time, can cause damage or death (infarction) of heart muscle tissue (myocardium).

Classical symptoms of acute myocardial infarction include sudden chest pain (typically radiating to the left arm or left side of the neck), shortness of breath, nausea, vomiting, palpitations, sweating, and anxiety (often described as a sense of impending doom). Among the diagnostic tests available to detect heart muscle damage are an electrocardiogram (ECG), echocardiography, and various blood tests. The most often used markers are the creatine kinase-MB (CK-MB) fraction and the troponin levels. Immediate treatment for suspected acute myocardial infarction includes oxygen, aspirin, and sublingual nitroglycerin.

Heart attacks are the leading cause of death for both men and women worldwide. Important risk factors include previous cardiovascular disease, older age, tobacco smoking, high blood levels of certain lipids (triglycerides, low-density lipoprotein) and low levels of high density lipoprotein (HDL), diabetes, high blood pressure, obesity, chronic kidney disease, heart failure, excessive alcohol consumption, the abuse of certain drugs (such as cocaine and methamphetamine), and chronic high stress levels.

There are two basic types of acute myocardial infarction. Transmural infarctions are associated with atherosclerosis involving a major coronary artery. It can be subclassified into anterior, posterior, or inferior. Transmural infarcts extend through the whole thickness of the heart muscle and are usually a result of complete occlusion of the area's blood supply. Subendocardial infarctions involve a small area in the subendocardial wall of the left ventricle, ventricular septum, or papillary muscles. Subendocardial infarcts are thought to result from locally decreased blood supply, possibly from a narrowing of the coronary arteries. The subendocardial area is farthest from the heart's blood supply and is more susceptible to this type of pathology.

Clinically, a myocardial infarction can be further subclassified into a ST elevation MI (STEMI) versus a non-ST elevation MI (non-STEMI) based on ECG changes. A 2007 consensus document classifies myocardial infarction into five main types:
Type 1—Spontaneous myocardial infarction related to ischaemia due to a primary coronary event such as plaque erosion and/or rupture, fissuring, or dissection
Type 2—Myocardial infarction secondary to ischaemia due to either increased oxygen demand or decreased supply, e.g. coronary artery spasm, coronary embolism, anaemia, arrhythmias, hypertension, or hypotension
Type 3—Sudden unexpected cardiac death, including cardiac arrest, often with symptoms suggestive of myocardial ischaemia, accompanied by presumably new ST elevation, or new LBBB, or evidence of fresh thrombus in a coronary artery by angiography and/or at autopsy, but death occurring before blood samples could be obtained, or at a time before the appearance of cardiac biomarkers in the blood
Type 4—Associated with coronary angioplasty or stents:
Type 4a—Myocardial infarction associated with PCI
Type 4b—Myocardial infarction associated with stent thrombosis as documented by angiography or at autopsy
Type 5—Myocardial infarction associated with CABG

C. Arterial Stenosis

1. Carotid Stenosis

Carotid stenosis is a narrowing or constriction of the inner surface (lumen) of the carotid artery, usually caused by atherosclerosis. The carotid artery is the large artery whose pulse can be felt on both sides of the neck under the jaw. It starts from the aorta as the common carotid artery, and at the throat it forks into the internal carotid artery and the external carotid artery. The internal carotid artery supplies the brain, and the external carotid artery supplies the face. This fork is a common site for atherosclerosis, an inflammatory buildup of plaque that can narrow the common or internal artery.

The plaque can be stable and asymptomatic, or it can be a source of embolization. Emboli (solid pieces) break off from the plaque and travel through the circulation to blood vessels in the brain. As the vessel gets smaller, they can lodge in the vessel wall and restrict blood flow to parts of the brain that that vessel supplies. This ischemia can either be temporary giving a transient ischemic attack, or permanent resulting in a thromboembolic stroke.

Transient ischemic attacks (TIAs) are a warning sign, and are often followed by severe permanent strokes, particularly within the first two days. TIAs by definition last less than 24 hours (and usually last a few minutes), and usually take the form of a weakness or loss of sensation of a limb or the trunk on one side of the body, or loss of sight (amaurosis fugax) in one eye. Less common symptoms are artery sounds (bruits), or ringing in the ear (tinnitis).

2. Renal Stenosis

Renal artery stenosis is the narrowing of the renal artery, most often caused by atherosclerosis or fibromuscular dysplasia. This narrowing of the renal artery can impede blood flow to the target kidney. Hypertension and atrophy of the affected kidney may result from renal artery stenosis, ultimately leading to renal failure if not treated.

Atherosclerosis is the predominant cause of renal artery stenosis in the majority of patients, usually those with a sudden onset of hypertension at age 50 or older. Fibromuscular dysplasia is the predominant cause in young patients, usually females under 40 years of age. A variety of other causes exist. These include arteritis, renal artery aneurysm, extrinsic compression (e.g., neoplasms), neurofibromatosis, and fibrous bands.

D. Vein/Arterial Graft Transplant

Veins and arteries are used by vascular surgeons for autotransplantation in coronary artery bypass operations. In such procedures, one major concern is post-operative inflammation, stenosis and blockage. While arterial grafts may be desired, vein grafts are more common, and preferred when many grafts are required, such as in a triple bypass or quadruple bypass.

The great saphenous vein (GSV) is the large (subcutaneous) superficial vein of the leg and thigh. The great saphenous vein is the conduit of choice for vascular surgeons, when available, for doing peripheral arterial bypass operations because it has superior long-term patency compared to synthetic grafts, human umbilical vein grafts or biosynthetic grafts. Often, it is used in situ after tying off smaller tributaries and stripping of the valves.

E. Atherosclerosis

Atherosclerosis (also known as arteriosclerotic vascular disease or ASVD) is a condition in which an artery wall thickens as the result of a build-up of fatty materials such as cholesterol. It is a syndrome affecting arterial blood vessels, a chronic inflammatory response in the walls of arteries, in large part due to the accumulation of macrophage white blood cells and promoted by low-density lipoproteins (plasma proteins that carry cholesterol and triglycerides) without adequate removal of fats and cholesterol from the macrophages by functional high density lipoproteins (HDL). It is commonly referred to as a hardening or furring of the arteries. It is caused by the formation of multiple plaques within the arteries. Atherosclerosis is a chronic disease that remains asymptomatic for decades.

The atheromatous plaque is divided into three distinct components:
the atheroma, which is the nodular accumulation of a soft, flaky, yellowish material at the center of large plaques, composed of macrophages nearest the lumen of the artery
underlying areas of cholesterol crystals
calcification at the outer base of older/more advanced lesions

Atherosclerotic lesions, or atherosclerotic plaques are separated into two broad categories: stable and unstable (also called vulnerable). The pathobiology of atherosclerotic lesions is very complicated but generally, stable atherosclerotic plaques, which tend to be asymptomatic, are rich in extracellular matrix and smooth muscle cells, while, unstable plaques are rich in macrophages and foam cells and the extracellular matrix separating the lesion from the arterial lumen (also known as the fibrous cap) is usually weak and prone to rupture. Ruptures of the fibrous cap, expose thrombogenic material, such as collagen to the circulation and eventually induce thrombus formation in the lumen. Upon formation, intraluminal thrombi can occlude arteries outright (i.e., coronary occlusion), but more often they detach, move into the circulation and eventually occlude smaller downstream branches causing thromboembolism (i.e., Stroke is often caused by thrombus formation in the carotid arteries). Apart from thromboembolism, chronically expanding atherosclerotic lesions can cause complete closure of the lumen. Interestingly, chronically expanding lesions are often asymptomatic until lumen stenosis is so severe that blood supply to downstream tissue(s) is insufficient resulting in ischemia.

These complications of advanced atherosclerosis are chronic, slowly progressive and cumulative. Most commonly, soft plaque suddenly ruptures (see vulnerable plaque), causing the formation of a thrombus that will rapidly slow or stop blood flow, leading to death of the tissues fed by the artery in approximately 5 minutes. This catastrophic event is called an infarction. One of the most common recognized scenarios is called coronary thrombosis of a coronary artery, causing myocardial infarction. Even worse is the same process in an artery to the brain, commonly called stroke. Another common scenario in very advanced disease is claudication from insufficient blood supply to the legs, typically due to a combination of both stenosis and aneurysmal segments narrowed with clots. Since atherosclerosis is a body-wide process, similar events occur also in the arteries to the brain, intestines, kidneys, legs, etc. Many infarctions involve only very small amounts of tissue and are termed clinically silent, because the person having the infarction does not notice the problem, does not seek medical help or when they do, physicians do not recognize what has happened.

F. Stent Placement

In medicine, a stent is an artificial tube or sleeve inserted into a natural passage/conduit in the body to prevent, or counteract, a disease-induced, localized flow constriction. The term may also refer to a tube used to temporarily hold such a natural conduit open to allow access for surgery. A coronary stent is a tube placed in the coronary arteries that supply the heart, to keep the arteries open in the treatment of coronary heart disease. It is used in a procedure called percutaneous coronary intervention (PCI). Stents reduce chest pain, but they have not been shown to improve survival, except in acute myocardial infarction. Similar stents and procedures are used in non-coronary vessels, e.g., in the legs in peripheral artery disease.

Treating a blocked (“stenosed”) coronary artery with a stent follows the same steps as other angioplasty procedures with a few important differences. The interventional cardiologist uses angiography to assess the location and estimate the size of the blockage (“lesion”) by injecting a contrast medium through the guide catheter and viewing the flow of blood through the downstream coronary arteries. Intravascular ultrasound (IVUS) may be used to assess the lesion's thickness and hardness (“calcification”). The cardiologist uses this information to decide whether to treat the lesion with a stent, and if so, what kind and size. Drug eluting stents are most often sold as a unit, with the stent in its collapsed form attached onto the outside of a balloon catheter. Outside the U.S., physicians may perform “direct stenting” where the stent is threaded through the lesion and expanded. Common practice in the U.S. is to predilate the blockage before delivering the stent. Predilation is accomplished by threading the lesion with an ordinary balloon catheter and expanding it to the vessel's original diameter. The physician withdraws this catheter and threads the stent on its balloon catheter through the lesion. The physician expands the balloon which deforms the metal stent to its expanded size. The cardiologist may “customize” the fit of the stent to match the blood vessel's shape, using IVUS to guide the work.

Coronary artery stents, typically a metal framework, can be placed inside the artery to help keep it open. However, as the stent is a foreign object (not native to the body), it incites an immune response. This may cause scar tissue (cell proliferation) to rapidly grow over the stent. In addition, there is a strong tendency for clots to form at the site where the stent damages the arterial wall. Since platelets are involved in the clotting process, patients must take dual antiplatelet therapy afterwards, usually clopidogrel and aspirin for one year and aspirin indefinitely. In order to reduce the treatment, a new generation of stent has been developed with biodegradable polymer.

However, the dual antiplatelet therapy may be insufficient to fully prevent clots that may result in stent thrombosis; these and the cell proliferation may cause the standard (“bare-metal”) stents to become blocked (restenosis). Drug-eluting stents were designed to lessen this problem; by releasing an antiproliferative drug (drugs typically used against cancer or as immunosuppressants), they can help avoid this in-stent restenosis (re-narrowing).

G. Combinations

Where standard therapies are available for any of the aforementioned disease states, one may apply such standard therapies in combination with the drug formulations disclosed herein, included but not limited to clopidogrel, aspirin/PPI or combinations thereof.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods for Study 1

Study Design and Subjects.

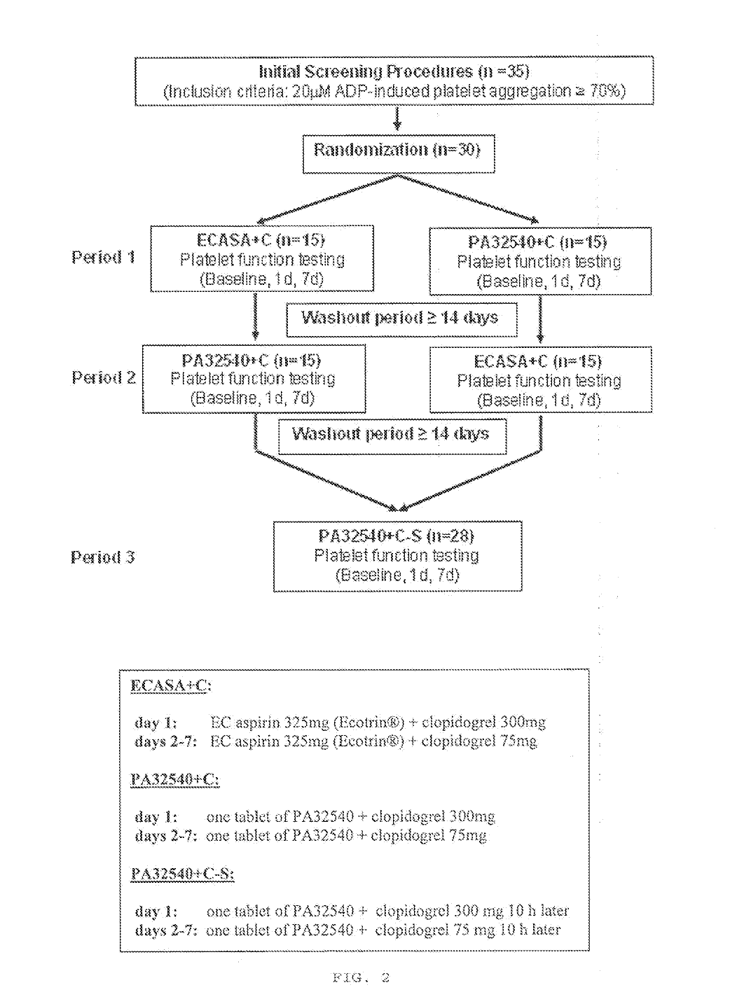

The SPACING study was a randomized, open-label, single-center, crossover study in healthy volunteers aged 40 or older. The study was performed in accordance with standard ethical principles; written consent was obtained from all patients. Exclusion criteria were subjects with a bleeding diathesis or a history of gastrointestinal bleeding, hemorrhagic stroke, illicit drug or alcohol abuse, coagulopathy, major surgery within 6 weeks prior to randomization, platelet count <100,000/mm³, hematocrit <25%, creatinine >4 mg/dL, elevated liver enzymes, or current use of NSAIDs, anticoagulants, or antiplatelet drugs other than aspirin. The study design is shown in FIG. 2.

Subjects were screened for eligibility if pre-therapy 20 μM adenosine diphosphate (ADP)-induced maximal aggregation was ≧70%. Thirty Subjects were then randomly assigned to receive each of the first two treatment regimens in a crossover fashion as follows: 300 mg clopidogrel+one 325 mg tablet of Ecotrin® on day 1 followed by 75 mg clopidogrel+one 325 mg tablet of Ecotrin® on days 2-7 (ECASA+C); or 300 mg clopidogrel+one tablet of PA32540 on day 1 followed by 75 mg clopidogrel+one tablet of PA32540 on days 2-7 (PA32540+C). During the first two treatment periods, a protocol amendment was finalized by the institutional review board to include a third treatment period. During day 1 of treatment period 3, subjects were administered one tablet of PA32540 in the morning+one tablet of 300 mg clopidogrel 10 hours later followed by one tablet of PA32540 in the morning+one tablet of 75 mg clopidogrel 10 hours later on days 2-7 (PA32540+C−S). There was a minimum washout period of 14 days between each treatment period.

Study Drug Administration and Protocol Compliance.

Study drug administration was performed only at the research unit under the supervision of site staff and included a mouth check to ensure that the study drug had been swallowed. Each dose of medication was administered with 240 mL of water. During synchronous therapy first clopidogrel was given followed immediately by aspirin or PA32540. Study subjects were provided breakfast and instructed not to eat until 1 hour after drug administration. Subjects were explicitly instructed by means of a written list not to consume food or liquids containing caffeine during the study. Compliance was supervised by study staff. After day 6, subjects were confined to the research unit until after day 7 procedures were complete to ensure strict adherence to the study protocol.

Blood and Urine Sampling.

Urine was analyzed for cocaine, cannabis, opiates, amphetamines, barbiturates, benzodiazepines and alcohol was determined by breath test at screening and at check-in on day 1 and on day 6 of each treatment period. All female subjects of childbearing potential were given a pregnancy test at screening and at check-in on day 1 of each period and no randomized subject had a positive result. A positive test result for alcohol, illicit drugs, or pregnancy would exclude the subject from participation in the study.

Pre-treatment blood samples were collected after overnight fast (≧10 hrs) and before morning dosing. At 24 hours and 7 days after assigned treatment, blood samples were collected after an overnight fast and 1 hour after clopidogrel administration. Blood was collected from the antecubital vein into Vacutainer® tubes (Becton-Dickinson, Franklin Lakes, N.J.) after discarding the first 2-3 mL of free flowing blood; the tubes were filled to capacity and gently inverted 3 to 5 times to ensure complete mixing of the anticoagulant. Tubes containing 3.2% trisodium citrate were used for light transmittance aggregometry and the vasodilator-stimulated phosphoprotein phosphorylation (VASP-P) assay. In addition, two tubes containing 3.2% sodium citrate (Greiner B10-One Vacuette North America, Inc., Monroe, N.C.) were collected for the VerifyNow P2Y12 and ASA assays.

Light Transmittance Aggregometry.

The blood-citrate tubes were centrifuged at 120 g for 5 minutes to recover platelet rich plasma and further centrifuged at 850 g for 10 minutes to recover platelet poor plasma. The platelet rich plasma and platelet poor plasma fractions were stored at room temperature to be used within 30 minutes. Platelet aggregation was assessed as described previously. Briefly, platelets were stimulated with 5 and 20 μM ADP, and 2 mM arachidonic acid (AA). Maximal aggregation (PAₘₐₓ) was assessed using a Chronolog Lumi-Aggregometer (Model 490-4D) with the Aggrolink software package (Chrono-log Corp, Havertown, Pa.) (Gurbel et al., 2009).

Vasodilator Stimulated Phosphoprotein-Phosphorylation Assay.

The measurement of VASP-P is a method of quantifying P2Y₁₂ receptor reactivity and reflects the extent of P2Y₁₂ receptor blockade. The platelet reactivity index (PRI) was calculated after measuring the VASP-P levels [mean fluorescence intensity (MFI)] determined by monoclonal antibodies following stimulation with prostaglandin (PGE₁) (MFI_{PGE1}) and also PGE1+ADP (MFI_{PGE1+ADP}) according to the commercially available Biocytex (Biocytex, Inc, Marseille, France) assay. The PRI (%) is calculated by the equation [(MFI_{PGE1})−(MFI_{PGE1+ADP})]/(MFI_{PGE1})×100% (Bonello et al., 2008).

VerifyNow-ASA and P2Y12 Assay.

The VerifyNow assay is a turbidimetric based optical detection system that measures platelet aggregation in whole blood (Price et al., 2008; Gurbel et al., 2007). The aspirin cartridge contains a lyophilized preparation of human fibrinogen-coated beads, arachidonic acid, preservative and buffer. The assay is designed to measure platelet function based upon the binding activated platelets to fibrinogen after stimulation. The instrument measures an optical signal, reported as aspirin reaction units (ARU). For the P2Y12 assay, ADP is used as the agonist, and platelet reactivity is reported as P2Y12 reaction units (PRU).

Endpoints.

The primary endpoint measure was relative inhibition of platelet aggregation (IPA) at day 7 defined as IPA (%)=[(PA₀−PA₇)/PA₀]×100 where PA₇ was the maximum 20 μM ADP-induced platelet aggregation (PA₂₀ₘₐₓ) at day 7 and PA₀ was the maximum 20 μM ADP-induced platelet aggregation at baseline.

A secondary endpoint was the IPA at day 7 using the 2 mM AA-induced maximum platelet aggregation (PA_{AA}). Other endpoints included IPA at day 7 measured by 5 μM ADP-induced maximum aggregation (PA₅ₘₐₓ), IPA from pre-dose to day 1 post-dose, and relative inhibition of baseline measurements of PRI, PRU, ARU. The absolute change from pre-dose to day 1 and from pre-dose to day 7 post-dose in PA₂₀ₘₐₓ (ΔPA₂₀ₘₐₓ), PA₅ₘₐₓ (ΔPA₅ₘₐₓ), PRI (ΔPRI), and PRU (ΔPRU), were also calculated.

Statistical Analysis and Sample Size Calculation.

This study required 30 subjects per treatment arm (15 per sequence in a crossover fashion). Using 2.5% one-sided test and 90% power the sample size was sufficient to reject the null hypothesis that PA32540+C is inferior to ECASA+C at a non-inferiority margin of 10%. The inventor prespecified that ECASA+C would be associated with a mean IPA of 40% at day 7 and a standard deviation of 12%. The sample size and power calculations were made under the assumption that non-inferiority would be tested with the expectation that the difference between ECASA+C and PA32540+C would be zero. The sample size also provided sufficient power to test the non-inferiority between PA32540+C−S and ECASA+C.

The primary analysis was to demonstrate the non-inferiority of PA32540+C or PA32540+C−S compared to ECASA+C. Non-inferiority was established if the upper bound of a two-sided 95% confidence interval for the treatment difference in least square means of IPA (Treatment A-Treatment B at day 7 or Treatment A-Treatment C at day 7) was ≦10% IPA.

Comparisons between ECASA+C versus PA32540+C for the relative change and the absolute change from baseline were performed using analysis of variance (ANOVA) for cross-over design. The ANOVA model included sequence, period, and treatment as fixed effects, and subject within sequence as a random effect. The 95% confidence intervals for the difference between treatment least-squares means (LSM) was calculated. The paired t-test was used to compare the treatment differences between PA32540+C−S and ECASA+C and also used to compare the differences between post-treatment timepoints. Statistical analyses were performed using SAS version 9.1 or higher (Cary, N.C.) and SPSS version 13 (SPSS Inc., Chicago, Ill.); p≦0.05 was considered statistically significant.

### Example 2

### Results for Study 1

Study Population.

Baseline demographics of the study cohort are shown in Table 1. Thirty healthy volunteers, with a mean age of 45 and a body mass index of 26 kg/m², were enrolled. Subjects were predominantly Caucasian. Thirty subjects completed the first 2 periods of the study, whereas 28 patients completed the final arm of the study. There were no serious adverse events reported throughout the study. Treatment-related adverse events were classified as ecchymosis (during ECASA+C=10, PA32540+C=9 and PA32540+C−S=7), gastrointestinal upset (during ECASA+C=1, PA32540+C=1), headache (during PA32540+C=1), and epistaxis (during PA32540+C=1).

Aspirin Effect.

There was no difference in pre-dose arachidonic acid-induced aggregation and ARUs between treatments (data not shown). Post-dose arachidonic acid-induced aggregation was low (3-7%) at 1 and 7 days after ECASA+C and PA32540+C dosing. IPA and ARU measurements did not differ between treatments at 24 hour post-loading and at day 7 (Tables 2 and 3).

Primary Analysis.

Synchronous administration of PA32540 with clopidogrel failed to meet the non-inferiority criterion whereas spaced administration met the non-inferiority definition (upper 95% CI for difference in least squared means=13.2% IPA vs. 9.6% IPA, respectively (Tables 2 and 3).

Light Transmittance Aggregometry.

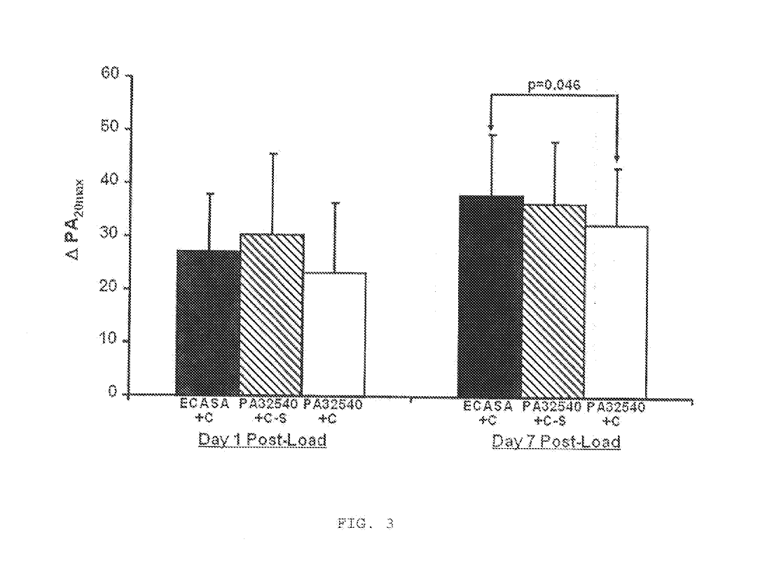

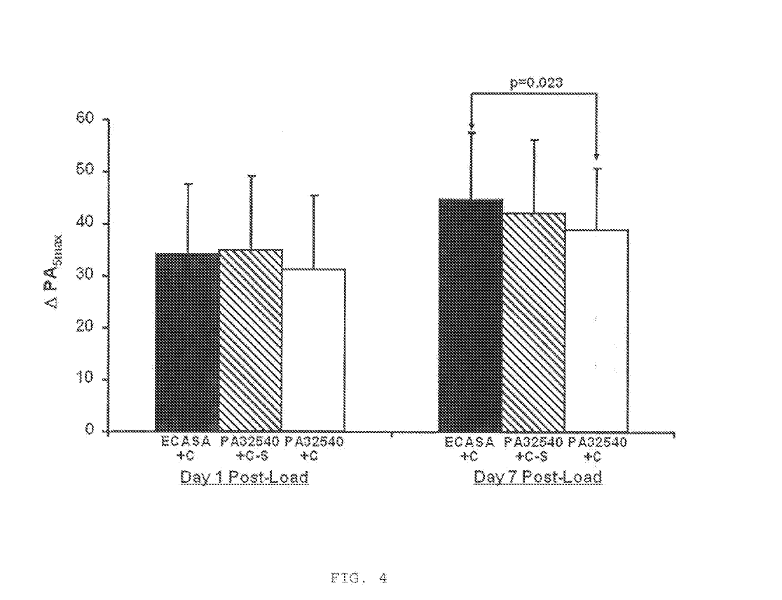

A reduced antiplatelet effect induced by omeprazole was most evident during maintenance therapy with synchronous PA32540 and clopidogrel administration (Tables 2 and 3). ΔPA₅ₘₐₓ and ΔPA₂₀ₘₐₓ increased from 1 to 7 days post-dosing (p<0.001 for all treatments (FIGS. 3 and 4). At day 1 post-dose, the IPA₂₀ₘₐₓ during PA32540+C−S was marginally higher than the IPA₂₀ₘₐₓ during in ECASA+C. However the IPA₂₀ₘₐₓ during PA32540+C and the IPA₅ₘₐₓ during PA32540+C−S and PA32540+C were lower than ECASA+C (Tables 2 and 3). ΔPA₂₀ₘₐₓ and ΔPA₅ₘₐₓ both increased by spacing clopidogrel therapy in subjects treated with PA32540 (FIGS. 3 and 4).

The VerifyNow-P2Y12 Assay.

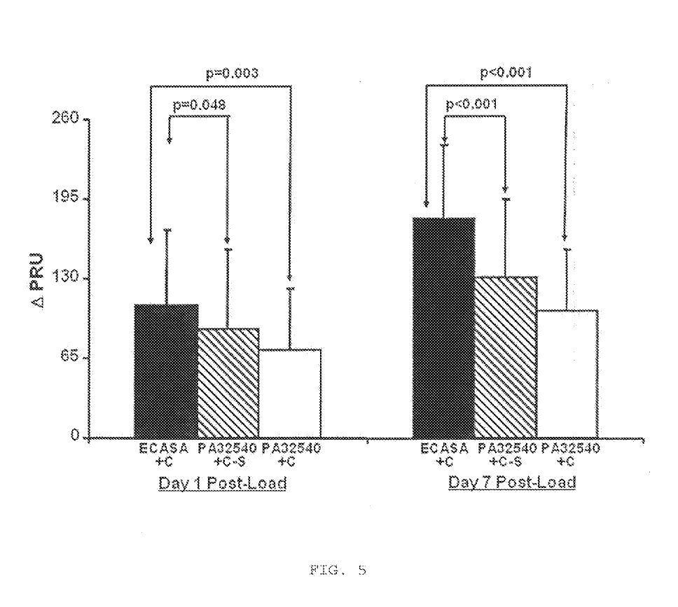

A similar attenuation in the omeprazole—clopidogrel interaction by drug spacing was observed by VerifyNow measurements (Tables 2 and 3; FIG. 5).

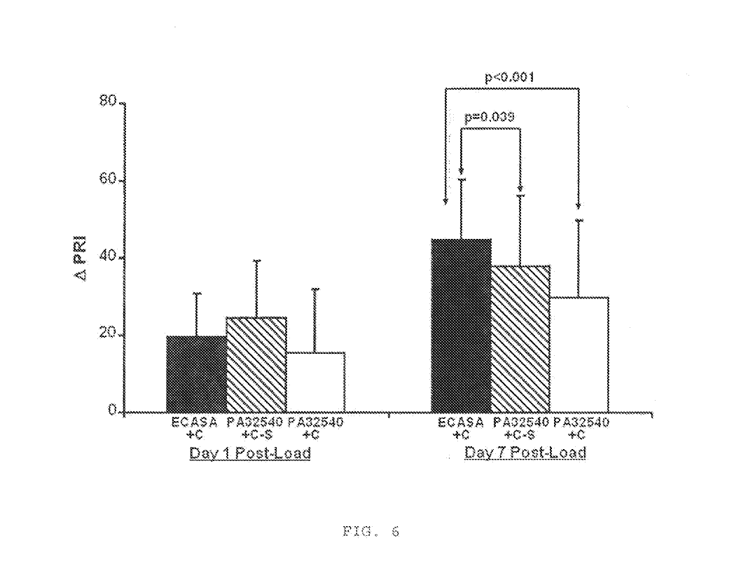

VASP-P assay. Similar to ΔPA₂₀ₘₐₓ and ΔPA₅ₘₐₓ, ΔPRI also increased by spacing clopidogrel therapy in subjects treated with PA32540 (p=0.05 at 1 and 7 days post-dose, FIG. 6). The attenuation in the clopidogrel—omeprazole interaction by spacing also was evidenced by examining the differences between groups in relative inhibition of baseline PRI as shown in Tables 2 and 3. At day 1 post-dosing, there was a 5.2% difference between ECASA+C versus PA32540+C in the relative inhibition of baseline PRI as compared to a −3.4% difference between ECASA+C versus PA32540+C−S. At day 7 the attenuation of the interaction by spacing also was evident. ΔPRI was greater at day 7 compared to day 1 post dosing in all groups (p<0.001).

TABLE 1
Demographics

| | Subjects | |
|---|---|---|
| | (n = 30) | |
| Age (years) | 45 ± 5 | |
| Male, n (%) | 12 | (40) |
| Body mass index, kg/m² | 26 ± 3 | |
| Race, n, (%) | | |
| Caucasian | 27 | (90) |
| African American | 1 | (3) |
| Asian | 2 | (7) |

| | |
|---|---|
| Laboratory Assessment | |
| White Blood Cells (×1000/mm³) | 5.9 ± 1.1 |
| Platelets (×1000/mm³) | 252 ± 51 |
| Hemoglobin (g/dL) | 13.7 ± 1.2 |
| Hematocrit (%) | 41.1 ± 3.3 |
| Creatinine (g/dL) | 0.8 ± 0.2 |

TABLE 2
Inhibition of Platelet Function During Synchronous Administration

| | | | |
|---|---|---|---|
| | | | Least Square |
| | | | Means |
| Endpoint | ECASA325 + C | PA32540 + C | Difference¹ |
| (mean) | (n = 30) | (n = 30) | (95% CI) |

At Day 1 Post-loading

| | | | |
|---|---|---|---|
| 2 mM AA- | 91.8 | 91.5 | 0.3 (−0.6, 1.2) |
| induced Aggrega- | | | |
| tion | | | |
| ARU | 34.0 | 34.5 | −0.5 (−2.7, 1.7) |
| 20 μM ADP- | 31.2 | 26.1 | 5.1 (0.3, 10.0) |
| induced Aggrega- | | | |
| tion | | | |
| 5 μM ADP | 41.4 | 36.7 | 4.7 (−1.2, 10.7) |
| VASP-PRI | 23.0 | 17.8 | 5.2 (−0.1, 10.3) |
| PRU | 33.3 | 23.4 | 9.9 (4.0, 15.9) |

At Day 7 Post-loading

| | | | |
|---|---|---|---|
| 2 mM AA- | 91.2 | 91.4 | −0.3 (−0.9, 0.4) |
| induced Aggrega- | | | |
| tion | | | |
| ARU | 34.5 | 36.4 | −1.9 (−6.0, 2.1) |
| 20 μM ADP- | 44.0 | 36.7 | 7.3 (1.4, 13.2) |
| induced Aggrega- | | | |
| tion² | | | |
| 5 μM ADP- | 54.0 | 45.9 | 8.1 (2.5, 13.7) |
| induced Aggrega- | | | |
| tion | | | |
| VASP-PRI | 52.8 | 34.5 | 18.3 (10.7, 26.0) |
| PRU | 56.1 | 32.8 | 23.4 (17.9, 28.8) |

ARU-Aspirin reaction units; PRU = P2Y12 reaction units, ADP = adenosine diphosphate; VASP-PRI = vasodilator stimulated phosphoprotein phosphorylation-platelet reactivity index
¹Negative values represent increase in % inhibition.
²Primary endpoint

TABLE 3
Inhibition of Platelet Function During Spacing Administration

| | | | |
|---|---|---|---|
| | | | Mean |
| | ECASA325 + C | PA32540 + C-S | Difference |
| Endpoint | (n = 28) | (n = 28) | (95% CI) |

At Day-1 Post-loading

| | | | |
|---|---|---|---|
| 20 μM ADP-induced Aggregation | 31.8 | 33.2 | −1.4 (−7.5, 4.8) |
| 5 μM ADP-induced Aggregation | 42.0 | 38.7 | 3.3 (−4.5, 11.1) |
| VASP-PRI | 23.3 | 26.7 | −3.4 (−8.6, 1.7) |
| PRU | 33.9 | 27.1 | 6.8 (0.6, 13.0) |

At Day 7 Post-loading

| | | | |
|---|---|---|---|
| 20 μM ADP-induced Aggregation¹ | 44.4 | 40.0 | 4.4 (−0.8, 9.6) |
| 5 μM ADP-induced Aggregation | 54.1 | 46.6 | 7.5 (0.9, 14.1) |
| VASP-PRI | 51.9 | 41.7 | 10.1 (3.6, 16.7) |
| PRU | 56.5 | 40.6 | 15.9 (9.9, 21.8) |

ARU—Aspirin reaction units;
PRU = P2Y12 reaction units,
ADP = adenosine diphosphate;
VASP-PRI = vasodilator stimulated phosphoprotein phosphorylation-platelet reactivity index
¹= Primary endpoint

### Example 3

### Discussion for Study 1

This is the first pharmacodynamic evaluation of the antiplatelet properties of PA32540, a novel combination product of 325 mg EC aspirin and 40 mg immediate-release omeprazole during synchronous and spaced administration following a clopidogrel loading dose of 300 mg and a maintenance 75 mg daily dose. The major findings of the present study are as follows: (1) a strategy of delayed administration of clopidogrel by 10 hours with PA32540 therapy attenuates the pharmacodynamic interaction caused by synchronous administration during loading and maintenance therapy as measured by multiple widely investigated methods; (2) the antiplatelet response measured after stimulation by arachidonic acid is the same after PA32540 and enteric coated aspirin administration; and (3) the omeprazole-clopidogrel interaction was most revealed by the VerifyNow P2Y12 assay and appeared to be most prominent during maintenance therapy.

Many studies have attempted to elucidate and establish the extent of the clinical interaction between clopidogrel and PPIs, particularly omeprazole (Gurbel et al., 2010). These studies have involved retrospective clinical outcome analyses. The Clopidogrel and the Optimization of Gastrointestinal Events (COGENT-1) trial is the only prospective randomized investigation that evaluated the clinical outcomes of patients treated with dual antiplatelet therapy with or without PPI therapy. In the COGENT-1 trial delayed-release 20 mg omeprazole was combined with 75 mg clopidogrel in a novel preparation (CGT-2168). COGENT-1 was prematurely terminated after enrollment of 3627 of 5000 planned patients (Siller-Matula et al., 2009). However, the available data suggested no difference in ischemic outcomes between patients treated with CGT-2168+enteric coated aspirin versus clopidogrel+enteric coated aspirin (Siller-Matula et al., 2009). Recently, Siller-Matula et al. performed a systematic review and meta-analysis of studies including 152,138 patients, and concluded that co-administration of PPI's and clopidogrel increased the risk of combined major cardiovascular events by 29% and the risk of myocardial infarction by 31%.³ However, PPI treatment decreased the risk of developing gastrointestinal bleeding by 50% (Bhatt et al., 2010).

Multiple pharmacodynamic studies have evaluated the PPI-clopidogrel interaction (Gurbel et al., 2010; Angiolillo et al., 2011; Ferreiro et al., 2010; Gilard et al., 2008; Sibbing et al., 2009; Würtz et al., 2010; Giraud et al., 1997). A reduced platelet inhibition measured by VASP-P in a PCI population during dual antiplatelet therapy randomly assigned to synchronous 20 mg daily omeprazole therapy was first reported by Gilard et al. (2008). In a cross-sectional observational study of 1,000 patients, consecutive patients under clopidogrel maintenance treatment and scheduled for a control coronary angiography, Sibbing et al. (2009) demonstrated that ADP-induced platelet aggregation measured with multiple electrode platelet aggregometry was significantly higher in patients treated with omeprazole (295.5 [193.5-571.2] AU*min) compared to patients without omeprazole treatment (220.0 [143.8−388.8] AU*min; p=0.001).²¹

Recently, Angiolillo et al. (2011) summarized the differential effects of 80 mg daily omeprazole on the pharmacodynamics of clopidogrel treatment (no aspirin therapy) (300 mg load/75 mg daily maintenance) in studies of healthy subjects in the absence of aspirin treatment. During clopidogrel therapy platelet aggregation and PRI significantly increased and IPA decreased irrespective of the timing of omeprazole administration. A similar study using the more common 40 mg dose of omeprazole in the absence of aspirin therapy demonstrated a reduction in antiplatelet effect when drugs were administered together or separately during the maintenance phase of treatment. However, platelet reactivity assessed by light transmittance aggregometry was higher during omeprazole therapy, but did not reach a threshold of statistical significance.

The results of previously published studies appear to be discordant with the attenuation in the interaction that the inventor observed with spaced administration of PA32540 and clopidogrel (Angiolillo et al., 2011; Ferreiro et al., 2010). This discordance may be explained by one or more of differences. In the SPACING study, the inventor selected the more commonly used lower dose of 40 mg rather than 80 mg omeprazole. If the interaction is due to the result of competitive inhibition at CYP2C19, lower plasma concentrations of omeprazole would produce less drug-drug interaction. PA32540 has an immediate-release omeprazole formulation with peak plasma levels at 30 minutes. The drug-drug interaction was observed at 1 day post-dose when dosed together but not when dosed separately. This observation suggests an immediate competitive inhibition since synchronous administration would lead to overlapping high plasma levels of omeprazole and clopidogrel (peak plasma levels at 30-60 minutes). But, with separate dosing omeprazole plasma levels are expected to be undetectable at the time of peak clopidogrel plasma levels at 1 day post-dose. At day 7 post-dose an effect on platelet aggregation was also observed when doses were administered together and less when doses were separate. In the SPACING study, subjects were treated with 325 mg aspirin which may have effects on ADP-induced platelet aggregation. In the previous studies of drug spacing, aspirin was excluded.

This study is discordant with previous studies demonstrating that omeprazole attenuates aspirin bioavailability, and the effect of aspirin on platelet aggregation (Würtz et al., 2010; Giraud et al., 1997). Here, the inventor found no difference in the antiplatelet effects measured by arachidonic acid stimulation in PA32540 versus ECASA treated subjects. A previous study by the inventor's group demonstrated greater reduction in urinary 11-dehydro thromboxane B₂ levels in subjects treated with PA32540 versus 81 mg enteric coated ASA (Gurbel et al., 2009).

The present study consisted of healthy volunteers ≧40 years of age; similar findings may not occur in the analysis of platelet function in patients with coronary artery disease. Secondly, the study did not assess pharmacokinetics, which may have elucidated a mechanism for the reduced interaction occurring after spaced therapy. Genotyping to determine CYP 2C19 loss-of-function and gain-of-function allele carrier status was not performed. Also, the inventor did not compare the antiplatelet response of clopidogrel between the immediate-release formulations of omeprazole in PA32540 and delayed-release omeprazole. Finally, similar to previous studies, the inventor only assessed the interaction for a short period of time. Extrapolation of these data to long-term effects would be highly speculative. Different pharmacodynamic effects of spaced therapy from those observed in the current study may occur in patients treated with other agents metabolized by the CYP2C19 pathway.

In conclusion, the inventor reports that the spacing of PA32540 and clopidogrel therapy significantly reduced the pharmacodynamic interaction observed during synchronous administration. Further studies evaluating a strategy that spaces PA32540 and clopidogrel therapy are warranted to confirm the inventor's observations.

### Example 4

### Materials and Methods for Study 2

Objectives:

The primary objective of this trial was to to evaluate adenosine diphosphate (ADP)-induced platelet aggregation following administration of clopidogrel, EC aspirin 81 mg and EC omeprazole 40 mg, all dosed concomitantly, and PA32540 and clopidogrel dosed separately. Secondarily, the goal was to evaluate arachidonic acid (AA)-induced platelet aggregation following administration of clopidogrel, EC aspirin 81 mg and EC omeprazole 40 mg, all dosed concomitantly, and PA32540 and clopidogrel dosed separately. Finally, the safety of each of the treatment arms was to be assessed.

Methodology:

This was a randomized, open-label, single-center, cross-over study in approximately 30 healthy subjects aged 40 or older. Study drugs were administered to each subject after being randomly assigned to receive each of the two treatment regimens in a two-way crossover fashion as follows:
Treatment A—AM dosing of one tablet of PA32540 followed approximately 10 hours later by clopidogrel 300 mg (Plavix® 300 mg) on Day 1, and then AM dosing of one tablet of PA32540 followed approximately 10 hours later by clopidogrel 75 mg (Plavix® 75 mg) on Days 2-7
Treatment B—clopidogrel 300 mg (Plavix® 300 mg)+one tablet of EC aspirin 81 mg (Bayer® 81 mg)+one capsule of EC omeprazole 40 mg (Prilosec® 40 mg) dosed concomitantly on Day 1, and clopidogrel 75 mg (Plavix® 75 mg)+one tablet of EC aspirin 81 mg (Bayer® 81 mg)+one capsule of EC omeprazole 40 mg (Prilosec® 40 mg) dosed concomitantly on Days 2-7
   The study design consisted of a screening period and two seven day treatment periods with a washout period of at least 14 days between periods.

Screening (Days −28 to −1):

After informed consent is obtained, subjects underwent assessments to qualify for study participation. Screening assessments consisting of a review of inclusion/exclusion criteria, medical history, ECG, clinical laboratory tests (hematology, chemistry and urinalysis), urine drug screen, a pregnancy test for women, physical exam including vitals signs and a review of concomitant medications were performed. A blood sample will be drawn to determine platelet aggregation (≧70% for eligibility) and CYP2C19 carrier testing. The assessments did not necessarily occur on the same day but prior to progressing to the study treatment period. No grapefruit or grapefruit juice could be ingested within the 10 days prior to dosing or during the study period.

Eligible subjects were instructed to abstain from alcohol consumption during the treatment period. Minimal alcohol consumption (no more than two units per day, on average, e.g., no more than two bottles of beer or no more than two glasses of wine) was allowed up until 48 hours prior to each treatment period. Subjects were also not allowed to drink any caffeinated beverages, or eat any dark chocolate for 48 hours prior to the Day 1 blood sample. Subjects were required to fast 10 hours prior to Day 1 blood sampling.

Day 1:

After at least a 10 hour overnight fast, concomitant medications were reviewed, adverse events were reviewed and recorded as appropriate, vital signs were recorded, and a urine drug screen and a pregnancy test for women was performed. Blood samples were obtained before the AM dosing for baseline platelet aggregation assessment Chronolog (20 μM ADP and 2 mM AA used separately as agonists). Subjects were randomly assigned to receive either Treatment A or Treatment B in the morning with 240 ml of water. Subjects were served a standard breakfast approximately one hour after dosing and released from the unit. Subjects on Treatment A returned to the Phase 1 unit in the PM to receive clopidogrel at least 10 hours later—approximately one hour prior to dinner.

Days 2-6:

Subjects reported to the Research unit each morning on an out-patient basis to receive the assigned treatment regimen with 240 ml of water. Subjects were served a standard breakfast approximately one hour after AM dosing and released from the unit.

Subjects on Treatment A returned to the Phase 1 unit in the PM to receive clopidogrel at least 10 hours later—approximately one hour prior to dinner. In the morning of Treatment Day 5, subjects were reminded not to drink any caffeinated beverages, or to eat any dark chocolate until after the Day 7 blood sampling. Concomitant medications were reviewed and adverse events recorded as appropriate. On Treatment Day 6, a urine drug screen was performed on all subjects.

Day 7:

Treatment A. After at least a 10 hour overnight fast, subjects received PA32540 with 240 ml of water in the morning and were served a standard breakfast approximately one hour after dosing. Approximately two hours after dosing, a blood sample was obtained for AA-induced platelet aggregation evaluation. Subjects returned to the Research unit for PM dosing of clopidogrel at least 10 hours after the AM dosing of PA32540 and approximately two hours later had a blood sample taken for ADP-induced platelet aggregation evaluation. Subjects were discharged after all study related procedures are completed.

Treatment B: After at least a 10 hour overnight fast, subjects received clopidogrel, EC aspirin 81 mg and EC omeprazole 40 mg all dosed concomitantly with 240 ml of water in the morning and served a standard breakfast approximately one hour after dosing. Approximately two hours after dosing, subjects had a blood sample taken for AA- and ADP-induced platelet aggregation evaluation. Subjects were discharged after all study related procedures were completed.

Washout Period:

There was at least a 14-day washout period between the last dose in Period 1 and the first dose in Period 2 where the above procedures (from Day 1) were repeated after subjects were crossed over to the other treatment regimen. Clinical adverse events were recorded and concomitant medications reviewed and recorded throughout this period.

End of Study Assessments:

Prior to discharge from the Research unit on Day 7 of treatment Period 2, the following procedures were completed: vital signs, blood draw for clinical laboratory analyses, urine collection for urinalysis, collection of adverse events and concomitant medications. These procedures were performed whenever a subject discontinued from the study prematurely.

Diagnosis and Main Criteria for Inclusion/Exclusion:

A subject was eligible for inclusion in this study if all of the following criteria applied:

1. Male or non-lactating, non-pregnant female subjects who are 40 years or older at the time of initial dosing.

2. Female subjects are eligible for participation in the study if they are of:
a) non-childbearing potential (i.e., physiologically incapable of becoming pregnant); or
b) childbearing potential, have a negative pregnancy test at Screening, and at least one of the following applies or is agreed to by the subject:
   Female sterilization or sterilization of male partner; or,
   Hormonal contraception by oral route, implant, injectable, vaginal ring; or,
   Any intrauterine device (IUD) with published data showing that the lowest expected failure rate is less than 1% per year;
   Double barrier method (2 physical barriers or 1 physical barrier plus spermicide); or
   Any other method with published data showing that the lowest expected failure rate is less than 1% per year

3. Physical status within normal limits for age and consistent with observations at screening.

4. Able to understand and comply with study procedures required and able and willing to provide written informed consent prior to any study procedures being performed.

A subject was not eligible for this study if any one or more of the following criteria applied:

1. History of hypersensitivity, allergy or intolerance to omeprazole or other proton-pump inhibitors (PPIs).

2. History of hypersensitivity, allergy or intolerance to aspirin or any NSAID and/or a history of NSAID-induced symptoms of asthma, rhinitis, and/or nasal polyps.

3. History of hypersensitivity or intolerance to clopidogrel.

4. History of hepatitis B or C, a positive test for hepatitis B surface antigen, hepatitis C antibody, a history of human immunodeficiency virus (HIV) infection or demonstration of HIV antibodies.

5. History of malignancy, treated or untreated, within the past five years, with the exception of successfully treated basal cell or squamous cell carcinoma of the skin.

6. Evidence of uncontrolled, or unstable cardio- or cerebrovascular disorder, which in the Investigator's opinion, would endanger a subject if he/she were to participate in the study.

7. Presence of an uncontrolled acute, or a chronic medical illness, e.g., GI disorder, diabetes, hypertension, thyroid disorder, bleeding disorder, infection, which in the Investigator's opinion would endanger a subject if he/she were to participate in the study or interfere with the objective of this study.

8. Schizophrenia or bipolar disorder.

9. GI disorder or surgery leading to impaired drug absorption.

10. Participation in any study of an investigational treatment in the 4 weeks before screening, or participation in another study at any time during this study.

11. <70% platelet aggregation at screening.

12. Donation of blood or plasma within 4 weeks of the study.

13. PPI use or any enzyme inducing/inhibiting agents within 4 weeks prior to dosing.

14. Body Mass Index outside the range of 19-32 kg/m² at screening.

15. Taking any medication(s) or nutritional supplement not approved by the Principle Investigator within 4 weeks of the first study drug administration and during the study.

16. Taking any antiplatelet drug within 2 weeks of the screening visit or during the study, or more than two 325 mg doses of aspirin or more than 2 doses of any other NSAIDs within 14 days prior to the screening visit.

17. Use of any tobacco product (including smoking cessation products containing nicotine) for at least three months prior to screening and during the treatment and washout periods.

18. History (in the past year) suggestive of alcohol or drug abuse or dependence, or excessive alcohol use (>2 units per day on average; for example, >2 bottles of beer, >2 glasses of wine) or use of alcohol as of 48 hours prior and during the treatment periods.

19. Any abnormal screening laboratory value that is clinically significant in the Investigator's opinion.

20. Any clinically significant abnormal baseline electrocardiogram (ECG).

21. Ingestion of grapefruit or grapefruit juice within 10 days of dosing or during the study.

22. Positive illicit drug screen.

23. Subjects who are in some way under the supervision of the principal investigator for this study.

24. Previous participation in another PA32540 clinical research trial.

Investigational Product, Dosage and Mode of Administration:

PA32540 (delayed release aspirin 325 mg plus immediate release omeprazole 40 mg) tablet administered orally once daily in the morning.

Duration of Treatment:

Two seven-day treatments with a 14-day washout period in between treatments.

Reference Therapy, Dosage and Mode of Administration:

Treatment A (PA32540 group)—
Clopidogrel (Plavix®) tablet, 10 hours post PA32540
one 300 mg loading dose in the PM of Day 1
one 75 mg maintenance dose in the PM of Days 2-7

Treatment B—
One EC aspirin (Bayer®) 81 mg tablet plus one EC omeprazole (Prilosec®) 40 mg capsule plus one Clopidogrel (Plavix®) tablet of 300 mg (loading dose) all taken concomitantly in the AM of Day 1.
One EC aspirin (Bayer®) 81 mg tablet plus one EC omeprazole (Prilosec®) 40 mg capsule plusone Clopidogrel (Plavix®) tablet of 75 mg (maintenance dose) all taken concomitantly in the AM of Days 2-7.

Criteria for Evaluation:

Efficacy: Platelet aggregation tests; chronolog using 20 μM ADP and 2 mM AA as agonists.

Safety: Vital signs, clinical laboratory tests and adverse events.

Sample Size: The sample size for this study was calculated using the statistical software nQuery Advisor version 6.0. A sample size of 30 subjects in each treatment (15 per sequence in a crossover fashion) has >90% power to detect a mean difference of 10 in inhibition of platelet aggregation (IPA) between PA32540 plus clopidogrel dosed separately and EC aspirin 81 mg plus EC omeprazole 40 mg plus clopidogrel dosed concomitantly using a two-sample t-test at 5% two-sided significance level assuming that the mean IPA of PA32540 plus clopidogrel dosed separately is 40 and the standard deviation of treatment differences is 14.

Analysis of Platelet aggregation: The endpoint measure was IPA defined as IPA (%)=[1−PA7/PA0]×100 where PA7 is the platelet aggregation (PA) at day 7 and PA0 is the platelet aggregation at baseline. The IPA was analyzed using analyses of variance (ANOVA). The ANOVA model included sequence, period and treatment as fixed effects, and subjects within sequence as a random effect. The mean differences of treatments were tested and p-values reported. The differences between treatment least-squares (LS) means and associated 95% confidence intervals were calculated.

Safety Analysis: Adverse events were coded using the MedDRA (Medical Dictionary for Regulatory Activities) and summarized for each treatment by SOC and preferred term. Tabulations and listings of values for vital signs and clinical laboratory tests were presented.

### Example 5

### Results for Study 2

As shown by the data that follow, PA32450 (enteric-coated aspirin 325 mg and immediate-release omeprazole 40 mg) given in conjunction with clopidogrel, dosed at least 10 hours apart, resulted in significantly better inhibition of ADP-induced platelet aggregation when compared to current standard of care (81 mg of enteric-coated aspirin, enteric-coated 40 mg omeprazole and clopidogrel). The improvement was approximately 20%. Tables 4-27 show the details of the study.

TABLE 4
Subject Disposition
All Randomized Subjects

| | Total | |
|---|---|---|
| End of Study | (N = 30) | |
| Safety Population | 30 | (100%) |
| ITT Population | 30 | (100%) |
| PP Population | 29 | (97%) |
| Completed Study | 29 | (97%) |
| Withdrawn Prematurely | 1 | (3%) |
| Primary Reason for Withdrawal | | |
| Adverse Event | 1 | (3%) |
| Lost to Follow-up | 0 | |
| Study Terminated by Sponsor | 0 | |
| Withdrew Consent | 0 | |
| Lack of Efficacy | 0 | |
| Other | 0 | |

TABLE 5
Demographics
Safety Population

| | | Total (N = 30) | |
|---|---|---|---|
| Age (years) | N | 30 | |
| | Mean (SD) | 49.3 | (5.7) |
| | Median | 49.5 | |
| | Min-Max | 40-62 | |
| Gender | N | 30 | |
| | Male | 13 | (43%) |
| | Female | 17 | (57%) |
| Race | N | 30 | |
| | White | 23 | (77%) |
| | Black/African American | 6 | (20%) |
| | Asian | 1 | (3%) |
| | American Indian or Alaska Native | 0 | |
| | Native Hawaiian or Other Pacific | 0 | |
| | Islander | | |
| Ethnic Origin | N = 30 | | |
| | Hispanic or Latino | 0 | |
| | Not Hispanic or Latino | 30 | (100%) |

TABLE 6
Demographics
Safety Population

| | | Total (N = 30) |
|---|---|---|
| Height (cm) | N | 30 |
| | Mean (SD) | 171.96 (10.05) |
| | Median | 170.82 |
| | Min-Max | 154.9-193.0 |
| Weight (kg) | N | 30 |
| | Mean (SD) | 79.38 (15.93) |
| | Median | 77.11 |
| | Min-Max | 50.8-115.7 |
| Body Mass Index (kg/m\hat{ }2) | N | 30 |
| | Mean (SD) | 26.675 (3.696) |
| | Median | 26.345 |
| | Min-Max | 19.22-32.00 |

TABLE 7
Medical History
Safety Population

| | Current | Past | |
|---|---|---|---|
| | Condition | Condition | |
| Medical Condition | (N = 30) | (N = 30) | |
| Blood and lymphatic system disorders | 0 | 0 | |
| Cardiovascular | 3 (10%) | 0 | |
| Congenital, familial and genetic disorders | 0 | 0 | |
| Ear and labyrinth disorders | 0 | 0 | |
| Endocrine disorders | 8 (27%) | 3 | (10%) |
| Eye disorders | 0 | 0 | |
| Gastrointestinal disorders | 0 | 2 | (7%) |
| Hepatobiliary disorders | 0 | 1 | (3%) |
| Immune system disorders | 5 (17%) | 0 | |
| Infection and infestations | 1 (3%) | 1 | (3%) |
| Injury, poisoning and procedural complications | 1 (3%) | 6 | (20%) |
| Metabolism and nutritional disorders | 1 (3%) | 0 | |
| Musculoskeletal & connective tissue disorders | 2 (7%) | 1 | (3%) |
| Neoplasms benign, malignant & unspecified | 0 | 1 | (3%) |
| (including cysts and polyps) | | | |
| Nervous System disorders | 3 (10%) | 0 | |
| Psychiatric disorders | 2 (7%) | 0 | |
| Renal and urinary disorders | 1 (3%) | 0 | |
| Reproductive system and breast disorders | 0 | 2 | (7%) |
| Respiratory, thoracic & mediastinal disorders | 2 (7%) | 1 | (3%) |
| Skin and subcutaneous tissue disorders | 0 | 0 | |
| Surgical and medical procedures | 0 | 19 | (63%) |
| Vascular disorders | 1 (3%) | 1 | (3%) |

TABLE 8
ECG at Screening
Safety Population

| | TOTAL | |
|---|---|---|
| Result | (N = 30) | |
| Normal | 22 | (73%) |
| Abnormal, not clinically significant | 8 | (27%) |
| Abnormal, clinically significant | 0 | |

TABLE 9
Concomitant Medications
Safety Population

| System Organ Class/ | Total |
|---|---|
| Preferred Term | (N = 30) |
| Subjects with Any Concomitant Medications | 9 (30%) |
| ANTIDEPRESSANTS | 3 (10%) |
| BUPROPION | 1 (3%) |
| CITALOPRAM HYDROBROMIDE | 1 (3%) |
| FLUOXETINE | 1 (3%) |
| PAROXETINE HYDROCHLORIDE | 1 (3%) |
| TRAZODONE | 1 (3%) |
| OTHER ANALGESICS AND ANTIPYRETICS | 3 (10%) |
| PARACETAMOL | 3 (10%) |
| THYROID PREPARATIONS | 2 (7%) |
| LEVOTHYROXINE SODIUM | 2 (7%) |
| ANTIHISTAMINES FOR SYSTEMIC USE | 1 (3%) |
| CETIRIZINE HYDROCHLORIDE | 1 (3%) |
| ANXIOLYTICS | 1 (3%) |
| LORAZEPAM | 1 (3%) |
| COUGH SUPPRESSANTS EXCL. COMB. WITH | 1 (3%) |
| EXPECTORANTS | |
| CODEINE | 1 (3%) |
| DRUGS AFFECTING BONE STRUCTURE AND | 1 (3%) |
| MINERALIZATION | |

TABLE 10
Concomitant Medications
Safety Population

| System Organ Class/ | Total |
|---|---|
| Preferred Term | (N = 30) |
| FOSAVANCE | 1 (3%) |
| OTHER UROLOGICALS, INCL. ANTISPASMODICS | 1 (3%) |
| DARIFENACIN | 1 (3%) |
| PSYCHOSTIM., AGENTS USED FOR ADHD AND | 1 (3%) |
| NOOTROPICS | |
| METHYLPHENIDATE HYDROCHLORIDE | 1 (3%) |
| VITAMIN A AND D, INCL. COMBINATIONS OF | 1 (3%) |
| THE TWO | |
| VITAMIN D NOS | 1 (3%) |

TABLE 11
Analysis of Percent Inhibition of Platelet Aggregation (IPA)
at Day 7 between Treatments A and B ITT Population

| | Treat- | | | | | | Mini- | Maxi- |
|---|---|---|---|---|---|---|---|---|
| Endpoint | ment | N | Mean | Std | Median | CV | mum | mum |
| 2 mM AA | A | 29 | 93.74 | 1.71 | 94.51 | 2 | 90.00 | 96.20 |
| | B | 30 | 90.09 | 20.48 | 95.12 | 23 | 0.00 | 98.78 |
| 20 μM ADP | A | 29 | 46.58 | 19.99 | 39.26 | 43 | 22.03 | 89.22 |
| | B | 30 | 39.37 | 19.38 | 38.62 | 49 | 4.94 | 74.59 |

Include baseline value in the model.
A = PA32540 + Clopidogrel (Dosed 10 hrs Apart)
B = EC Aspirin 81 mg + EC Omeprazole 40 mg + Clopidogrel

TABLE 12
Analysis of Percent Inhibition of Platelet Aggregation (IPA)
at Day 7 between Treatments A and B ITT Population

| | | | | LSMean | | | |
|---|---|---|---|---|---|---|---|
| | LSMean (SE) | | | Difference | 95% CI | | |
| Endpoint | A | B | Comparison | (SE) | Lower | Upper | p-value |
| 2 mM AA | 91.86 (1.27) | 92.06 (1.25) | A − B | −0.21 (1.66) | −3.61 | 3.19 | 0.901 |
| 20 μM ADP | 46.50 (3.55) | 39.25 (3.53) | A − B | 7.24 (2.27) | 2.57 | 11.91 | 0.004 |

Include baseline value in the model.
A = PA32540 + Clopidogrel (Dosed 10 hrs Apart)
B = EC Aspirin 81 mg + EC Omeprazole 40 mg + Clopidogrel

TABLE 13
Analysis of Percent Inhibition of Platelet Aggregation
(IPA) at Day 7 between Treatments A and B PP Population

| | Treat- | | | | | | Mini- | Maxi- |
|---|---|---|---|---|---|---|---|---|
| Endpoint | ment | N | Mean | Std | Median | CV | mum | mum |
| 2 mM AA | A | 29 | 93.74 | 1.71 | 94.51 | 2 | 90.00 | 96.20 |
| | B | 29 | 89.95 | 20.83 | 95.24 | 23 | 0.00 | 98.78 |
| 20 μM ADP | A | 29 | 46.58 | 19.99 | 39.26 | 43 | 22.03 | 89.22 |
| | B | 29 | 39.89 | 19.51 | 39.55 | 49 | 4.94 | 74.59 |

Include baseline value in the model.
A = PA32540 + Clopidogrel (Dosed 10 hrs Apart)
B = EC Aspirin 81 mg + EC Omeprazole 40 mg + Clopidogrel

TABLE 14
Analysis of Percent Inhibition of Platelet Aggregation
(IPA) at Day 7 between Treatments A and B PP Population

| | | | | LSMean | | | |
|---|---|---|---|---|---|---|---|
| | LSMean (SE) | | | Difference | 95% CI | | |
| Endpoint | A | B | Comparison | (SE) | Lower | Upper | p-value |
| 2 mM AA | 91.92 (1.25) | 91.86 (1.25) | A − B | 0.05 (1.65) | −3.32 | 3.43 | 0.975 |
| 20 μM ADP | 46.86 (3.62) | 39.69 (3.62) | A − B | 7.17 (2.28) | 2.48 | 11.85 | 0.004 |

Include baseline value in the model.
A = PA32540 + Clopidogrel (Dosed 10 hrs Apart)
B = EC Aspirin 81 mg + EC Omeprazole 40 mg + Clopidogrel

TABLE 15
Incidence of All Adverse Events - Safety Population

| System Organ Class/Preferred Term | A (N = 29) | | B (N = 30) | |
|---|---|---|---|---|
| Subjects with Any Adverse Event | 14 | (48%) | 16 | (53%) |
| Nervous system disorders | 7 | (24%) | 5 | (17%) |
| Headache | 4 | (14%) | 5 | (17%) |
| Dizziness | 3 | (10%) | 0 | |
| Dysgeusia | 1 | (3%) | 0 | |
| Skin and subcutaneous tissue disorders | 6 | (21%) | 4 | (13%) |
| Ecchymosis | 6 | (21%) | 4 | (13%) |
| Gastrointestinal disorders | 3 | (10%) | 6 | (20%) |
| Flatulence | 2 | (7%) | 3 | (10%) |
| Constipation | 0 | | 2 | (7%) |
| Abdominal pain upper | 1 | (3%) | 0 | |
| Dyspepsia | 0 | | 1 | (3%) |
| Nausea | 0 | | 1 | (3%) |

A = PA32540 + Clopidogrel (Dosed 10 hrs Apart)
B = EC Aspirin 81 mg + EC Omeprazole 40 mg + Clopidogrel

TABLE 16
Incidence of All Adverse Events
Safety Population

| | A | B |
|---|---|---|
| System Organ Class/Preferred Term | (N = 29) | (N = 30) |
| Infections and infestations | 2 (7%) | 2 (7%) |
| Upper respiratory tract infection | 1 (3%) | 2 (7%) |
| Nasopharyngitis | 1 (3%) | 0 |
| General disorders and administration site | 2 (7%) | 0 |
| conditions | | |
| Feeling abnormal | 1 (3%) | 0 |
| Thirst | 1 (3%) | 0 |
| Cardiac disorders | 1 (3%) | 0 |
| Tachycardia | 1 (3%) | 0 |
| Eye disorders | 0 | 1 (3%) |
| Conjunctival haemorrhage | 0 | 1 (3%) |
| Metabolism and nutrition disorders | 0 | 1 (3%) |
| Decreased appetite | 0 | 1 (3%) |
| Reproductive system and breast disorders | 0 | 1 (3%) |
| Menorrhagia | 0 | 1 (3%) |
| Respiratory, thoracic and mediastinal disorders | 1 (3%) | 0 |
| Cough | 1 (3%) | 0 |

A = PA32540 + Clopidogrel (Dosed 10 hrs Apart)
B = EC Aspirin 81 mg + EC Omeprazole 40 mg + Clopidogrel

TABLE 17
Incidence of Serious Adverse Events
Safety Population

| | | |
|---|---|---|
| | A | B |
| System Organ Class/Preferred Term | (N = 29) | (N = 30) |

There were no Serious Adverse Events reported in this study
A = PA32540 + Clopidogrel (Dosed 10 hrs Apart)
B = EC Aspirin 81 mg + EC Omeprazole 40 mg + Clopidogrel

TABLE 18
Incidence of Treatment-Related Adverse Events
Safety Population

| System | A | B | |
|---|---|---|---|
| Organ Class/Preferred Term | (N = 29) | (N = 30) | |
| Subjects with Any Adverse Event | 8 (28%) | 10 | (33%) |
| Skin and subcutaneous tissue | 6 (21%) | 4 | (13%) |
| disorders | | | |
| Ecchymosis | 6 (21%) | 4 | (13%) |
| Gastrointestinal disorders | 3 (10%) | 4 | (13%) |
| Flatulence | 2 (7%) | 3 | (10%) |
| Abdominal pain upper | 1 (3%) | 0 | |
| Dyspepsia | 0 | 1 | (3%) |
| Nausea | 0 | 1 | (3%) |
| Eye disorders | 0 | 1 | (3%) |
| Conjunctival haemorrhage | 0 | 1 | (3%) |
| Metabolism and nutrition disorders | 0 | 1 | (3%) |
| Decreased appetite | 0 | 1 | (3%) |

A = PA32540 + Clopidogrel (Dosed 10 hrs Apart)
B = EC Aspirin 81 mg + EC Omeprazole 40 mg + Clopidogrel

TABLE 19
Incidence of Treatment-Related Adverse Events
Safety Population

| | A | B |
|---|---|---|
| System Organ Class/Preferred Term | (N = 29) | (N = 30) |
| Reproductive system and breast disorders | 0 | 1 (3%) |
| Menorrhagia | 0 | 1 (3%) |

A = PA32540 + Clopidogrel (Dosed 10 hrs Apart)
B = EC Aspirin 81 mg + EC Omeprazole 40 mg + Clopidogrel

TABLE 20
Incidence of Adverse Events by Maximum Severity
Safety Population

| | Preferred Term | | | | | |
|---|---|---|---|---|---|---|
| | A (N = 29) | | | B (N = 30) | | |
| | | Mod- | Se- | | Mod- | Se- |
| System Organ Class | Mild | erate | vere | Mild | erate | vere |
| Subjects with Any | 14 (48%) | 0 | 0 | 16 (53%) | 0 | 0 |
| Adverse Event [1] | | | | | | |
| Nervous system | 7 (24%) | 0 | 0 | 5 (17%) | 0 | 0 |
| disorders | | | | | | |
| Headache | 4 (14%) | 0 | 0 | 5 (17%) | 0 | 0 |
| Dizziness | 3 (10%) | 0 | 0 | 0 | 0 | 0 |
| Dysgeusia | 1 (3%) | 0 | 0 | 0 | 0 | 0 |
| Skin and sub- | 6 (21%) | 0 | 0 | 4 (13%) | 0 | 0 |
| cutaneous tissue | | | | | | |
| disorders | | | | | | |
| Ecchymosis | 6 (21%) | 0 | 0 | 4 (13%) | 0 | 0 |
| Gastrointestinal | 3 (10%) | 0 | 0 | 6 (20%) | 0 | 0 |
| disorders | | | | | | |
| Flatulence | 2 (7%) | 0 | 0 | 3 (10%) | 0 | 0 |
| Constipation | 0 | 0 | 0 | 2 (7%) | 0 | 0 |
| Abdominal pain | 1 (3%) | 0 | 0 | 0 | 0 | 0 |
| upper | | | | | | |
| Dyspepsia | 0 | 0 | 0 | 1 (3%) | 0 | 0 |
| Nausea | 0 | 0 | 0 | 1 (3%) | 0 | 0 |
| Infections and | 2 (7%) | 0 | 0 | 2 (7%) | 0 | 0 |
| infestations | | | | | | |
| Upper respiratory | 1 (3%) | 0 | 0 | 2 (7%) | 0 | 0 |
| tract infection | | | | | | |
| Nasopharyngitis | 1 (3%) | 0 | 0 | 0 | 0 | 0 |
| General disorders | 2 (7%) | 0 | 0 | 0 | 0 | 0 |
| and administration | | | | | | |
| site conditions | | | | | | |
| Feeling abnormal | 1 (3%) | 0 | 0 | 0 | 0 | 0 |
| Thirst | 1 (3%) | 0 | 0 | 0 | 0 | 0 |

A = PA32540 + Clopidogrel (Dosed 10 hrs Apart)
B = EC Aspirin 81 mg + EC Omeprazole 40 mg + Clopidogrel

TABLE 21
Incidence of Adverse Events by Maximum Severity
Safety Population

| | Preferred Term | | | | | |
|---|---|---|---|---|---|---|
| | A (N = 29) | | | B (N = 30) | | |
| | | Mod- | Se- | | Mod- | Se- |
| System Organ Class | Mild | erate | vere | Mild | erate | vere |
| Cardiac disorders | 1 (3%) | 0 | 0 | 0 | 0 | 0 |
| Tachycardia | 1 (3%) | 0 | 0 | 0 | 0 | 0 |
| Eye disorders | 0 | 0 | 0 | 1 (3%) | 0 | 0 |
| Conjunct. haemor. | 0 | 0 | 0 | 1 (3%) | 0 | 0 |
| Metabolism & | 0 | 0 | 0 | 1 (3%) | 0 | 0 |
| nutrition Disorders | | | | | | |
| Decreased appetite | 0 | 0 | 0 | 1 (3%) | 0 | 0 |
| Reproductive | 0 | 0 | 0 | 1 (3%) | 0 | 0 |
| system & breast | | | | | | |
| disorders | | | | | | |
| Menorrhagia | 0 | 0 | 0 | 1 (3%) | 0 | 0 |
| Respiratory, | 1 (3%) | 0 | 0 | 0 | 0 | 0 |
| thoracic & | | | | | | |
| mediastinal | | | | | | |
| disorders | | | | | | |
| Cough | 1 (3%) | 0 | 0 | 0 | 0 | 0 |

A = PA32540 + Clopidogrel (Dosed 10 hrs Apart)
B = EC Aspirin 81 mg + EC Omeprazole 40 mg + Clopidogrel

TABLE 22
Blood Chemistry Laboratory Results
Safety Population

| | | | | | Me- | | |
|---|---|---|---|---|---|---|---|
| | Visit | N | Mean | SD | dian | Min | Max |
| ALT | Screening | 30 | 34.77 | 15.40 | 28.50 | 19.00 | 75.00 |
| (Units/L) | Final Visit | 30 | 33.40 | 16.44 | 26.50 | 19.00 | 91.00 |
| AST | Screening | 30 | 22.53 | 8.87 | 21.00 | 11.00 | 45.00 |
| (Units/L) | Final Visit | 30 | 21.00 | 7.96 | 20.50 | 7.00 | 44.00 |
| Alkaline | Screening | 30 | 64.33 | 19.93 | 64.00 | 28.00 | 101.00 |
| Phosphatase | Final Visit | 30 | 66.97 | 21.62 | 62.00 | 32.00 | 125.00 |
| (Units/L) | | | | | | | |
| BUN | Screening | 30 | 16.00 | 3.38 | 16.00 | 7.00 | 22.00 |
| (mg/dL) | Final Visit | 30 | 15.57 | 4.19 | 14.50 | 9.00 | 25.00 |
| Chloride | Screening | 30 | 103.97 | 1.97 | 103.50 | 99.00 | 108.00 |
| (mmol/L) | Final Visit | 30 | 103.53 | 1.76 | 104.00 | 99.00 | 107.00 |
| Creatinine | Screening | 30 | 0.83 | 0.14 | 0.80 | 0.65 | 1.24 |
| (mg/dL) | Final Visit | 30 | 0.81 | 0.16 | 0.77 | 0.60 | 1.21 |

TABLE 23
Blood Chemistry Laboratory Results
Safety Population

| | | | | | Me- | | |
|---|---|---|---|---|---|---|---|
| | Visit | N | Mean | SD | dian | Min | Max |
| Glucose | Screening | 30 | 84.10 | 9.35 | 86.50 | 63.00 | 98.00 |
| (fasting) | Final Visit | 30 | 86.47 | 19.24 | 83.50 | 59.00 | 129.00 |
| (mg/dL) | | | | | | | |
| Potassium | Screening | 30 | 4.33 | 0.25 | 4.30 | 3.90 | 5.10 |
| (mmol/L) | Final Visit | 30 | 4.28 | 0.28 | 4.30 | 3.80 | 5.10 |
| Sodium | Screening | 30 | 138.53 | 1.66 | 138.50 | 136.00 | 141.00 |
| (mmol/L) | Final Visit | 30 | 138.13 | 1.28 | 138.00 | 135.00 | 140.00 |
| Total | Screening | 30 | 0.59 | 0.30 | 0.50 | 0.30 | 1.40 |
| Bilirubin | Final Visit | 30 | 0.50 | 0.28 | 0.40 | 0.20 | 1.30 |
| (mg/dL) | | | | | | | |

TABLE 24
Hematology Laboratory Results
Safety Population

| | | | | | Me- | | |
|---|---|---|---|---|---|---|---|
| | Visit | N | Mean | SD | dian | Min | Max |
| Basophils (%) | Screening | 30 | 0.54 | 0.35 | 0.40 | 0.10 | 1.80 |
| | Final Visit | 30 | 0.49 | 0.23 | 0.50 | 0.20 | 1.10 |
| Eosinophils (%) | Screening | 30 | 2.43 | 1.43 | 1.95 | 0.60 | 6.80 |
| | Final Visit | 30 | 2.78 | 2.10 | 2.20 | 1.00 | 11.70 |
| Hematocrit (%) | Screening | 30 | 41.30 | 3.43 | 41.05 | 35.00 | 48.70 |
| | Final Visit | 30 | 41.03 | 3.27 | 41.10 | 34.20 | 46.50 |
| Hemoglobin | Screening | 30 | 13.97 | 1.36 | 13.75 | 11.60 | 16.50 |
| (g/dL) | Final Visit | 30 | 13.77 | 1.30 | 13.80 | 11.00 | 15.90 |
| Lymphocytes (%) | Screening | 30 | 32.20 | 7.28 | 32.80 | 21.10 | 48.30 |
| | Final Visit | 30 | 29.60 | 7.61 | 30.40 | 17.90 | 46.40 |
| MCH (pg) | Screening | 30 | 30.37 | 1.52 | 30.40 | 26.50 | 32.90 |
| | Final Visit | 30 | 30.22 | 1.54 | 30.40 | 26.30 | 33.10 |
| MCHC (%) | Screening | 30 | 33.79 | 0.91 | 33.80 | 32.00 | 35.30 |
| | Final Visit | 30 | 33.57 | 0.95 | 33.70 | 31.60 | 35.30 |

TABLE 25
Hematology Laboratory Results
Safety Population

| | | | | | Me- | | |
|---|---|---|---|---|---|---|---|
| | Visit | N | Mean | SD | dian | Min | Max |
| MCV | Screening | 30 | 89.85 | 3.61 | 90.60 | 80.80 | 96.90 |
| (FL) | Final Visit | 30 | 90.01 | 3.59 | 89.80 | 81.20 | 97.30 |
| Mono- | Screening | 30 | 8.07 | 2.35 | 7.70 | 4.00 | 14.30 |
| cytes (%) | Final Visit | 30 | 6.96 | 2.22 | 6.95 | 2.90 | 12.70 |
| Neutro- | Screening | 30 | 56.76 | 8.02 | 58.05 | 36.20 | 68.70 |
| phils (%) | Final Visit | 30 | 60.16 | 7.57 | 61.65 | 45.60 | 72.70 |
| Platelets | Screening | 30 | 236.87 | 51.59 | 234.00 | 153.00 | 355.00 |
| (K/MM3) | Final Visit | 30 | 229.33 | 59.29 | 221.00 | 152.00 | 367.00 |
| RBC | Screening | 30 | 4.60 | 0.40 | 4.59 | 3.72 | 5.50 |
| (M/MM3) | Final Visit | 30 | 4.56 | 0.38 | 4.61 | 3.70 | 5.27 |
| WBC | Screening | 30 | 6.19 | 1.63 | 5.78 | 4.19 | 9.69 |
| (K/MM3) | Final Visit | 30 | 6.05 | 1.39 | 6.24 | 3.40 | 8.83 |

TABLE 26
Urinalysis Laboratory Test Results
Safety Population

| | Screening | | Final Visit | |
|---|---|---|---|---|
| Result | (N = 30) | | (N = 30) | |
| Glucose | | | | |
| 3+ | 1 | (3%) | 0 | |
| Negative | 29 | (97%) | 30 | (100%) |
| Microscopic Blood | | | | |
| 1 | 4 | (13%) | 3 | (10%) |
| 2 | 1 | (3%) | 4 | (13%) |
| 3 | 1 | (3%) | 1 | (3%) |
| 4 | 0 | | 1 | (3%) |
| <1 | 8 | (27%) | 6 | (20%) |
| Negative | 16 | (53%) | 15 | (50%) |
| Protein | | | | |
| Negative | 25 | (83%) | 26 | (87%) |
| Trace | 5 | (17%) | 4 | (13%) |

TABLE 27
Vital Signs
Safety Population

| | | | |
|---|---|---|---|
| Screening | A | B | Final Visit |
| (N = 30) | (N = 29) | (N = 30) | (N = 30) |

Heart Rate (beats/minute)

| | | | | |
|---|---|---|---|---|
| N | 30 | 29 | 30 | 30 |
| Mean | 71.1 (9.8) | 69.6 (8.1) | 71.0 (10.1) | 70.9 (12.2) |
| (SD) | | | | |
| Median | 69.5 | 68.0 | 70.0 | 67.5 |
| Min-Max | 55-92 | 59-89 | 54-97 | 58-121 |

Systolic Blood Pressure (mmHg)

| | | | | |
|---|---|---|---|---|
| N | 30 | 29 | 30 | 30 |
| Mean | 124.2 (10.4) | 119.7 (10.0) | 121.2 (12.0) | 124.7 (11.3) |
| (SD) | | | | |
| Median | 121.5 | 119.0 | 120.5 | 124.0 |
| Min-Max | 105-151 | 100-138 | 94-159 | 99-148 |

Diastolic Blood Pressure (mmHg)

| | | | | |
|---|---|---|---|---|
| N | 30 | 29 | 30 | 30 |
| Mean | 71.8 (8.3) | 69.3 (9.5) | 69.4 (10.7) | 71.3 (10.5) |
| (SD) | | | | |
| Median | 73.5 | 72.0 | 69.5 | 71.5 |
| Min-Max | 55-85 | 50-83 | 50-88 | 50-88 |

A = PA32540 + Clopidogrel (Dosed 10 hrs Apart)
B = EC Aspirin 81 mg + EC Omeprazole 40 mg + Clopidogrel

The foregoing description is considered as illustrative only of the principles of the invention. Further, since numerous modifications and changes will readily occur to those skilled in the art, it is not desired to limit the invention to the exact construction and process as described above. Accordingly, all suitable modifications and equivalents may be resorted to falling within the scope of the invention as defined by the claims that follow. The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

### REFERENCES

The following references, to the extent that they provide exemplary procedural or other details supplementary to those set forth herein, are specifically incorporated herein by reference.
U.S. Pat. No. 6,926,907
Angiolillo et al., Clin. Pharmacol. Ther., 89(1):65-74, 2011.
Bhatt et al., J. Am. Coll. Cardiol., 52:1502-17, 2008.
Bhatt et al., N Engl. J. Med., 363:1909-17, 2010.
Bonello et al., J. Am. Coll. Cardiol., 51:1404-1411, 2008.
Ferreiro et al., Circ. Cardiovasc. Interv., 3:436-41, 2010.
Fort et al., In: A Novel Combination of Delayed Release (DR) Aspirin (ASA) and Immediate-Release (IR) Omeprazole, is Associated with a Decreased Risk of Gastroduodenal Mucosal Injury, American College of Gastroenterology, Orlando, Fl, Poster 354, 2008.
Gilard et al., J. Am. Coll. Cardiol., 251:256-260, 2008.
Giraud et al., Aliment Pharmacol. Ther., 11:899-906, 1997.
Gurbel and Tantry, Nat. Rev. Cardiol., 8(1):7-8, 2011.
Gurbel et al., Circulation, 115:3156-64, 2007.
Gurbel et al., Circulation, 120:2577-85, 2009.
Gurbel et al., In: Safer Aspirin Therapy with Greater Thromboxane Suppression, Intl. Soc. Thrombosis and Haemostasis, MA, No 3120, 2009.
Gurbel et al., Drug, Healthcare and Patient Safety, 2:233-240, 2010.
King et al., J. Am. Coll. Cardiol., 51:172-209, 2008.
Laine and Hennekens, Am. J. Gastroenterol., 105:34-41, 2010.
Moukarbel et al., Eur Heart J., 30:2226-32, 2009.
Panara et al., Br. J. Pharmacol., 116:2429-2434, 1995.
Price et al., Eur. Heart J., 29:992-1000, 2008.
Remington's Pharmaceutical Sciences, 16^{th} Ed., Oslo (Ed.), Easton, Pa., 1980.
Sibbing et al., Thromb. Haemost., 101:714-9, 2009.
Siller-Matula et al., Am. Heart J., 157:148.e1-5, 2009.
Wakatani et al., Jpn. J. Pharmacol., 78:365-371, 1998.
Wiirtz et al., Heart, 96:368-71, 2010.

1. A method of providing an antiplatelet therapy to a subject in need thereof comprising co-administering to said subject a proton pump inhibitor (PPI) and clopidogrel such that:
(a) said clopidogrel and said omeprazole are delivered in a sequential manner;
(b) said clopidogrel is released (i) prior to or (ii) prior to and after said PPI; and
(c) said PPI achieves a peak plasma concentration at least 1 hour after a first clopidogrel peak plasma concentration.

2. The method of claim 1, wherein clopidogrel is delivered in multiple pulses.

3. The method of claims 2, wherein the number of clopidogrel pulses is 2, 3 or 4.

4. The method of claim 1, wherein said PPI achieves a peak plasma concentration at least 2 hours prior to a second, third or fourth clopidrogrel pulse.

5-6. (canceled)

7. The method of claim 1, wherein said subject is further administered aspirin.

8. The method of claim 7, wherein said aspirin is formulated for enteric release.

9. The method of claim 1, wherein said clopidogrel and said PPI are coformulated in a single drug formulation.

10. The method of claim 9, wherein said single drug formulation further comprises aspirin.

11. The method of claim 10, wherein said single drug formulation comprises an aspirin core, a PPI layer surrounding said aspirin core, and a clopidogrel layer surrounding said PPI layer.

12. The method of claim 1, wherein said clopidogrel and said PPI are formulated individually but administered at the same time.

13. The method of claim 2, wherein said PPI achieves a peak plasma concentration at least 2 hours after the first clopidogrel peak plasma concentration.

14. (canceled)

15. The method of claim 1, wherein said clopidogrel is pulsed twice at about 37.5 mg per pulse.

16. The method of claim 1, wherein said clopidogrel is pulsed three times at about 25 mg per pulse.

17. The method of claim 1, wherein said PPI is selected from the group consisting of omeprazole, lansoprazole, pantoprazole, esomeprazole, rabeprazole, and dexlansoprazole.

18. The method of claim 1, wherein said PPI is omeprazole.

19. The method of claim 1, wherein aspirin is coformulated with said omeprazole, wherein said omeprazole is delivered prior to said aspirin.

20. The method of claim 1, wherein said subject suffers from or is at risk of stroke, heart attack, arterial stenosis or atherosclerosis, or has or will undergo vein graft transplant or stent placement.

21. A drug formulation comprising:
(a) clopidogrel, wherein clopidrogrel is released immediately; and
(b) a proton pump inhibitor (PPI), wherein said PPI is released subsequent to said clopidogrel.

22-59. (canceled)

60. A method of treating a subject with an antiplatelet therapy comprising administering to said subject:
(a) enteric-coated aspirin coformulated with immediate-release omeprazole; and
(b) clopidogrel,
wherein (a) and (b) are dosed at least 10 hours apart.

61-64. (canceled)
